SEQUENCE LISTING (12 sequences)

SEQ ID NO: 1 - DNA, 66 bp, synthetic construct
  misc_feature 1..66: note = PCR1 forward primer 1
  source 1..66: mol_type = other DNA
acactctttccctacacgacgctcttccgatctgtagccggctttatatatcttgtggaaaggacg

SEQ ID NO: 2 - DNA, 66 bp, synthetic construct
  misc_feature 1..66: note = PCR1 forward primer 2
  source 1..66: mol_type = other DNA
acactctttccctacacgacgctcttccgatcttacaagggctttatatatcttgtggaaaggacg

SEQ ID NO: 3 - DNA, 59 bp, synthetic construct
  misc_feature 1..59: note = PCR1 reverse primer
  source 1..59: mol_type = other DNA
gtgactggagttcagacgtgtgctcttccgatctactgacgggcaccggagccaattcc

SEQ ID NO: 4 - DNA, 58 bp, synthetic construct
  misc_feature 1..58: note = PCR2 forward primer
  source 1..58: mol_type = other DNA
aatgatacggcgaccaccgagatctacactctttccctacacgacgctcttccgatct

SEQ ID NO: 5 - DNA, 64 bp, synthetic construct
  misc_feature 1..64: note = PCR2 reverse primer
  source 1..64: mol_type = other DNA
caagcagaagacggcatacgagattggtcagtgactggagttcagacgtgtgctcttccgatct

SEQ ID NO: 6 - DNA, 20 bp, synthetic construct
  misc_feature 1..20: note = sgRNA target 1
  source 1..20: mol_type = other DNA
ctggtctattactacgcccg

SEQ ID NO: 7 - DNA, 20 bp, synthetic construct
  misc_feature 1..20: note = sgRNA target 2
  source 1..20: mol_type = other DNA
tgggctgtgtctgaacgccg

SEQ ID NO: 8 - DNA, 20 bp, synthetic construct
  misc_feature 1..20: note = PCR and sequencing primer 1
  source 1..20: mol_type = other DNA
gatgagctgggctacaggtg

SEQ ID NO: 9 - DNA, 20 bp, synthetic construct
  misc_feature 1..20: note = PCR and sequencing primer 2
  source 1..20: mol_type = other DNA
gtcgtaagacgcccagacac

SEQ ID NO: 10 - DNA, 20 bp, synthetic construct
  misc_feature 1..20: note = sgRNA CD276
  source 1..20: mol_type = other DNA
cacagggcaacgcatccctg

SEQ ID NO: 11 - DNA, 20 bp, synthetic construct
  misc_feature 1..20: note = sgRNA control
  source 1..20: mol_type = other DNA
gtattactgatattggtggg

SEQ ID NO: 12 - protein, 10 aa, synthetic construct
  REGION 1..10: note = MART-1 epitope
ELAGIGILTV

(19) United States
(12) Patent Application Publication
(10) Pub. No.: US 2025/0281610 A1
(43) Pub. Date: Sep. 11, 2025

(54) P2RY2 ACTIVITY MODULATORS

(71) Applicants: Deutsches Krebsforschungszentrum Stiftung des öffentlichen Rechts, Heidelberg (DE); Stichting Het Nederlands Kanker Instituut-Antoni van Leeuwenhoek, Amsterdam (NL)

(72) Inventors: Chong SUN, Heidelberg (DE); Zhaoqing HU, Heidelberg (DE); Antonius Nicolaas Maria SCHUMACHER, Amsterdam (NL); Meike Emma Willemijn LOGTENBERG, Amsterdam (NL)

(73) Assignees: Deutsches Krebsforschungszentrum Stiftung des öffentlichen Rechts, Heidelberg (DE); Stichting Het Nederlands Kanker Instituut-Antoni van Leeuwenhoek, Amsterdam (NL)

(21) Appl. No.: 18/861,181

(22) PCT Filed: Apr. 28, 2023
(86) PCT No.: PCT/EP2023/061309

§ 371 Date: Oct. 28, 2024

(30) Foreign Application Priority Data
Apr. 29, 2022 (EP) 22170938.9

(51) Int. Cl. A61K 40/11; A61K 31/513; A61K 31/7105; A61K 38/47; A61K 39/00; A61K 39/395; A61K 40/31; A61K 40/32; A61K 40/42; A61P 35/00; C12N 15/11

(52) U.S. Cl. CPC A61K 40/11; A61K 31/513; A61K 31/7105; A61K 38/47; A61K 39/39558; A61K 40/31; A61K 40/32; A61K 40/421; A61K 40/4266; A61K 40/4272; A61P 35/00; C12N 15/11; A61K 2039/505; C12N 2310/20

(57) ABSTRACT

The present invention relates to a P2Y purinoceptor 2 (P2RY2) activity modulator for use in T cell immunotherapy. The present invention further relates to a polynucleotide encoding a P2RY2 activity modulator and to a host cell comprising the P2RY2 activity modulator for use in T cell immunotherapy. Furthermore, the present invention relates to a method of identifying 5 a subject amenable to T cell immunotherapy comprising (A) determining in a sample of said subject the activity of P2RY2; (B) comparing the activity determined in step (A) to a reference; and identifying a subject amenable to T cell immunotherapy based on the comparison of step (B), as well as to a method for identifying a P2RY2 activity modulator, said method comprising (I) contacting a host cell with a candidate compound suspected to be a P2RY2 activity 10 modulator; (II) determining B7-H3 activity in said host cell; (III) comparing the B7-H3 activity determined in step (II) to a control; and (IV) identifying a P2RY2 activity modulator based on the comparison in step (III).

The present invention relates to a P2Y purinoceptor 2 (P2RY2) activity modulator for use in T cell immunotherapy. The present invention further relates to a polynucleotide encoding a P2RY2 activity modulator and to a host cell comprising the P2RY2 activity modulator for use in T cell immunotherapy. Furthermore, the present invention relates to a method of identifying a subject amenable to T cell immunotherapy comprising (A) determining in a sample of said subject the activity of P2RY2; (B) comparing the activity determined in step (A) to a reference; and identifying a subject amenable to T cell immunotherapy based on the comparison of step (B), as well as to a method for identifying a P2RY2 activity modulator, said method comprising (I) contacting a host cell with a candidate compound suspected to be a P2RY2 activity modulator; (II) determining B7-H3 activity in said host cell; (III) comparing the B7-H3 activity determined in step (II) to a control; and (IV) identifying a P2RY2 activity modulator based on the comparison in step (III).

Therapies that promote functions or activities of immune cells to combat cancer have been transforming cancer treatment over recent years. In particular, engineered T cell therapies such as CAR-T and immune checkpoint blocking therapies have been shown to produce more durable responses than conventional treatments (e.g. chemotherapy) in a number of cancer types. However, current cancer immunotherapies are only effective in some patients and identification of treatment strategies that benefit more patients remains a challenge.

The success of targeting the PD-1/PD-L1 axis and the CTLA-4 immune checkpoint has stimulated interest in other molecules that may enhance the activity of existing cancer immunotherapies, or show activity in tumor types in which blockade of CTLA-4 and the PD-1/PD-L1 pathway shows lower activity. An immune checkpoint ligand of particular interest is the B7 family member B7-H3 (CD276). Similar to other B7 family members, B7-H3 has immune modulating functions, but its receptor on immune cells remains unknown. B7-H3 was initially identified as a co-stimulatory molecule that augments T cell activation in vitro and in autoimmune disease models, (Chen et al., J Immunol 189, 347-355 (2012)). However, subsequent work demonstrated that B7-H3 suppresses the effector function of both T cells and NK cells in vitro and in syngeneic mouse cancer models (Lee et al., Cell Res 27, 1034-1045 (2017); Cai et al., Cell Mol Immuno 17(3), 227-236 (2020)). In addition, an immunosuppressive activity of B7-H3 has also been described in mouse models of graft-versus-host disease (Veenstra et al., Blood 125, 3335-3346 (2015), cardiac allograft rejection (Ueno et al., Eur J 5 Immunol 42, 2343-2353 (2012), and autoimmune encephalomyelitis (Suh et al., Nat Immunol 4, 899-906 (2003); Prasad et al., J Immunol 73(4) 2500-2506 (2004)). Thus, the targeting of B7-H3 activity may be used to modulate immune cell (e.g. T cell and NK cell) function or activity.

Higher levels of B7-H3 expression in cancer cells compared to healthy tissue have been reported in many solid human tumor types, including non-small-cell lung cancer (NSCLC), breast cancer, ovarian cancer, endometrial cancer, lung cancer, liver cancer and gastric cancer, and in hematological tumors, including diffuse large B cell lymphoma (DLBCL), acute myeloid leukemia (AML) and mantle cell lymphoma (MCL). In solid tumors, B7-H3 expression has also been detected on surrounding tumor blood vessels and tumor-infiltrating immune cells, including myeloid cells (Yim et al., Eur J Cancer 133, 74-85 (2020)).

P2RY2 has previously been identified as a receptor of extracellular ATP and UTP (Soltoff et al., J Biol Chem 273 (5) 2653-2660 (1998); Burnstock et al., Purigergic Signal 9, 491-540 (2013)). Also, P2RY2 expression was found to correlate with patient survival in pancreatic ductal adenocarcinoma (Hu et al., Clin Cancer Res 25 (4): 1318-1330 (2019)).

Thus there is still a need for improved cancer treatments which provide advantageous options for the detection, treatment, and management of cancers. The technical problem underlying the present invention can be seen as the provision of means and methods for complying with the aforementioned needs. The technical problem is solved by the embodiments characterized in the claims and herein below.

In accordance, the present invention relates to a P2Y purinoceptor 2 (P2RY2) activity modulator for use in T cell immunotherapy.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof, thus, e.g. identifying “a cell” relates to identifying at least one cell, preferably to identifying a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods. Method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the polynucleotides and polypeptides, may be comprised in larger structures, e.g. may be covalently or non-covalently linked to further sequences, carrier molecules, retardants, and other excipients. In particular, polypeptides as specified may be comprised in fusion polypeptides comprising further peptides, which may serve e.g. as a tag for purification and/or detection, as a linker, or to extend the in vivo half-life of a compound. The term “detectable tag” refers to a stretch of amino acids which are added to or introduced into the fusion polypeptide; preferably, the tag is added C- or N-terminally to the fusion polypeptide. Said stretch of amino acids preferably allows for detection of the polypeptide by an antibody which specifically recognizes the tag; or it preferably allows for forming a functional conformation, such as a chelator; or it preferably allows for visualization, e.g. in the case of fluorescent tags. Preferred detectable tags are the Myc-tag, FLAG-tag, 6-His-tag, HA-tag, GST-tag or a fluorescent protein tag, e.g. a GFP-tag. These tags are all well known in the art. Other further peptides preferably comprised in a fusion polypeptide comprise further amino acids or other modifications which may serve as mediators of secretion, as mediators of blood-brain-barrier passage, as cell-penetrating peptides, and/or as immune stimulants. Further polypeptides or peptides to which the polypeptides may be fused are signal and/or transport sequences, e.g. an IL-2 signal sequence, and linker sequences.

The term “polypeptide”, as used herein, refers to a molecule consisting of several, typically at least 20, amino acids that are covalently linked to each other by peptide bonds. Molecules consisting of less than 20 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. Preferably, the polypeptide is comprised in a fusion polypeptide and/or a polypeptide complex. Unless specifically indicated otherwise, reference to specific polypeptides herein preferably includes polypeptide variants.

As used herein, the term “polypeptide variant” relates to any chemical molecule comprising at least one polypeptide as specified herein, having the indicated activity, but differing in structure from said specific polypeptide. Preferably, the polypeptide variant comprises a polypeptide having a contiguous amino acid sequence corresponding to at least 50%, preferably at least 75%, more preferably at least 85%, even more preferably at least 90%, most preferably at least 95%, of the amino acid sequence of the polypeptide specifically indicated. Moreover, it is to be understood that a polypeptide variant as referred to in accordance with the present invention shall have an amino acid sequence which differs due to at least one amino acid substitution, deletion and/or addition, wherein the amino acid sequence of the variant is still, preferably, at least 70%, more preferably at least 80%, even more preferably at least 90%, even more preferably at least 95%, still more preferably at least 98%, most preferably at least 99%, identical with the amino acid sequence of the specific polypeptide. The degree of identity between two amino acid sequences can be determined by algorithms well known in the art and as described herein above. Polypeptide variants referred to above may be allelic variants or any other species specific homologs, paralogs, or orthologs. Moreover, the polypeptide variants referred to herein include fragments of the specific polypeptides or the aforementioned types of polypeptide variants as long as these fragments and/or variants have the biological activity as specified. Such fragments may be or may be derived from, e.g., degradation products or splice variants of the polypeptides. Further included are variants which differ due to posttranslational modifications such as phosphorylation, glycosylation, ubiquitinylation, sumoylation, or myristylation, by including non-natural amino acids, and/or by being peptidomimetics.

The term “polynucleotide”, as used herein, refers to a linear or circular nucleic acid molecule. The polynucleotide of the present invention shall be provided, preferably, either as an isolated polynucleotide (i.e. isolated from its natural context) or in genetically modified form, preferably comprising at least one heterologous sequence. The term encompasses single- as well as double-stranded polynucleotides. Moreover, comprised are also chemically modified polynucleotides including naturally occurring modified polynucleotides such as glycosylated or methylated polynucleotides or artificially modified derivatives such as biotinylated polynucleotides, locked nucleic acids, and the like. The polynucleotides of the invention have the activity of encoding or being a P2RY2 activity modulator. Methods for testing whether a given polynucleotide has the aforesaid activity are known in the art. E.g., preferably, a candidate polynucleotide of interest may be introduced into a host cell and the resulting amount and/or activity of P2RY2 is determined, preferably in comparison to a control. Unless specifically indicated otherwise, reference to specific polynucleotides herein preferably includes polynucleotide variants.

The term “polynucleotide variant”, as used herein, relates to a variant of a polynucleotide referred to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide. Preferably, said polynucleotide variant is an ortholog, a paralog or another homolog of the specific polynucleotide. Also preferably, said polynucleotide variant is or is derived from a non-naturally occurring allele of the specific polynucleotide. Polynucleotide variants also encompass polynucleotides comprising a nucleic acid sequence which is capable of hybridizing to the aforementioned specific polynucleotides, preferably, under stringent hybridization conditions. These stringent conditions are known to the skilled worker and can be found in Current Protocols in Molecular Biology, John Wiley & Sons, N. Y. (1989), 6.3.1-6.3.6. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide of the present invention. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, plants or, preferably, from animals may be used. Further, variants include polynucleotides comprising nucleic acid sequences which are at least 70%, preferably at least 80%, more preferably at least 90%, even more preferably at least 95%, still more preferably at least 98%, most preferably at least 99%, identical to the specifically indicated nucleic acid sequences. Moreover, also encompassed are polynucleotides which comprise nucleic acid sequences encoding amino acid sequences which are at least 70%, more preferably at least 80%, even more preferably at least 90%, even more preferably at least 95%, still more preferably at least 98, most preferably at least 99%, identical to the amino acid sequences specifically indicated. The percent identity values are, preferably, calculated over the entire amino acid or nucleic acid sequence region, preferably as specified herein above. The polynucleotides of the present invention either consist, essentially consist of, or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Also, the polynucleotide may be comprised in a vector.

The term “vector”, preferably, encompasses phage, plasmid, viral or retroviral vectors as well artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a host. The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells. More preferably, in the vector of the invention the polynucleotide is operatively linked to expression control sequences allowing expression in prokaryotic or eukaryotic cells or isolated fractions thereof, i.e. preferably, the polynucleotide is comprised in an expression vector. Expression of said polynucleotide comprises transcription of the polynucleotide into an RNA, preferably as specified above. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA1, pcDNA3 (InVitrogene) or pSPORT1 (GIBCO BRL). Preferably, said vector is an expression vector and a gene transfer or targeting vector. Expression vectors derived from viruses such as retroviruses, vaccinia virus, adeno-associated virus, herpes viruses, or bovine papilloma virus, may be used for delivery of polynucleotides or vectors into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The term “P2Y purinoceptor 2”, which may be abbreviated as “P2RY2”, is known to the skilled person to relate to a member of the group of G-protein coupled receptors, which is responsive to both adenosine and uridine nucleotides. Preferably, P2RY2 is a mammalian P2RY2, more preferably a human P2RY2, most preferably having an amino acid sequence as shown in Genbank Acc. No: NP_002555.4, NP_788085.3, or NP_788086.3, preferably encoded by a nucleic acid sequence as shown in Genbank Acc. No: NM_002564.4, NM_176071.3, or NM_176072.3. Preferably, the P2RY2 is a P2RY2 expressed by a cancer cell or by a T cell mediating autoimmune disease.

The term “activity modulator” is known to the skilled person to relate to any compound causing the activity of a biological molecule or pathway to deviate, preferably significantly, from the activity in the absence of said activity modulator. Preferably, said deviation is a deviation of at least 20%, more preferably at least 50%, even more preferably at least 75%, even more preferably at least 90% of the value of an activity parameter determinable in the absence of said activity modulator, in particular in case the activity modulator is an activity decreasing compound; said deviation may, however, also be a deviation by a factor of at least two, preferably at least five, more preferably at least ten, in particular in case the activity modulator is an activity increasing compound. As is understood by the skilled person in view of the description herein below, modulation may, however, also be complete abolishment of an activity present in the absence of said modulator; or may be de novo activity not present in the absence of the modulator. The effect of the activity modulator may be temporary, e.g. over a time frame of hours or days, or may be permanent, in particular depending on the specific choice of the modulator. Preferably, said effect is temporary and lasts for of from 1 day to 6 months, preferably of from 2 days to 2 months, more preferably of from 3 days to 4 weeks, most preferably of from 1 to 4 weeks. As will also be understood by the skilled person, the effect may be local, i.e. topical at a site of administration, or may be systemic, e.g. after systemic administration of the activity modulator.

Preferably, the activity modulator is an activity decreasing compound; thus, preferably the P2RY2 activity modulator is a P2RY2 activity decreasing compound, preferably a P2RY2-specific activity decreasing compound. Preferably, the P2RY2-specific activity decreasing compound inhibits non-P2RY2-specific activity by at most 50%, preferably at most 25%, more preferably by at most 10% at a concentration inhibiting P2RY2 activity by 90%. The activity of a P2RY2 activity decreasing compound is, preferably, determined in vitro by assaying the amount and/or enzymatic activity of P2RY2 as specified elsewhere herein, preferably as shown herein in the Examples. Also preferably, said P2RY2 activity decreasing compound increases T cell anti-cancer activity in vitro and/or in vivo. Compounds decreasing activity of a known polypeptide gene product such as P2RY2 can be provided by the skilled person by standard methods of molecular biology, e.g. as specified herein below. Preferably, the P2RY2 activity modulator is a P2RY2 activity decreasing compound in case the T cell immunotherapy is cancer T cell immunotherapy.

Preferably, the P2RY2 activity decreasing compound is a direct P2RY2 activity decreasing compound, i.e. a compound binding to, preferably specifically binding to, and thereby inhibiting P2RY2. More preferably, the direct P2RY2 activity decreasing compound is a small molecule inhibitor, an inhibitor polypeptide, an inhibitor polynucleotide, or a non-polypeptide non-polynucleotide inhibitor macromolecule. Preferably, the direct P2RY2 activity decreasing compound is a compound binding to at least one epitope in P2RY2, preferably an epitope including at least one amino acid of an interaction epitope and/or an active center of P2RY2. The skilled person is aware of methods suitable for determining binding of a direct inhibitor to P2RY2, e.g. staining of P2RY2-positive cells or of extracts from such cells with a candidate direct inhibitor, wherein said inhibitor is coupled to a detectable label, preferably a colored and/or fluorescent dye; ELISA methods; surface plasmon resonance methods, and the like.

The term “small molecule”, as used herein, relates to a chemical molecule with a molecular mas of at most 2.5 kDa, preferably at most 2 kDa, more preferably at most 1.5 kDa, most preferably at most 1 kDa. The “small molecule inhibitor” may, in principle, be from any chemical class of molecules. Preferably, the small molecular inhibitor is an organic molecule, i.e. comprises at least one carbon-carbon bond. Small molecule inhibitors of P2RY2 activity are known in the art, e.g from WO 1999/002501, in particular. AR-C 118925XX (CAS No: 216657-60-2), and can be identified by a method as specified herein below.

The term “inhibitor polypeptide” is used herein to relate to any and all polypeptides or peptides binding to P2RY2 and inhibiting its activity. Thus, the inhibitor polypeptide preferably is an antagonist, more preferably a competitive antagonist or a non-competitive antagonist. Preferably, the inhibitor polypeptide is selected from the list consisting of an antibody, an aptamer, an anticalin, and a Designed Ankyrin Repeat Protein (DARPin).

As used herein, the term “antibody” relates to a soluble immunoglobulin from any of the classes IgA, IgD, IgE, IgG, or IgM, or fragments thereof, having the activity of directly interacting with P2RY2 and inhibiting P2RY2 activity as specified herein above. Antibodies against P2RY2 or fragments thereof can be prepared by well-known methods using a purified P2RY2 polypeptide or a suitable fragment derived therefrom as an antigen. A fragment which is suitable as an antigen may be identified by antigenicity determining algorithms well known in the art. Suitable fragments may also be obtained either from the P2RY2 polypeptide by proteolytic digestion, may be synthetic peptides, or may be recombinantly expressed. Suitability of an antibody thus generated as an inhibitor of P2RY2 can be tested by an assay as described elsewhere herein. Preferably, the antibody of the present invention is a monoclonal antibody, a human, primatized, chimerized, or humanized antibody, or a fragment thereof. More preferably, the antibody is a single chain antibody, a single-domain antibody, a nanobody, or an antibody fragment, such as Fab, scFab, and the like. Also comprised as antibodies of the present invention are a bispecific antibody, a synthetic antibody, or a chemically modified derivative of any of the aforesaid antibodies. Preferably, the antibody of the present invention shall specifically bind (i.e. does not cross react with other polypeptides or peptides) to a P2RY2 polypeptide as specified above. Specific binding can be tested by various well-known techniques. Antibodies or fragments thereof can be obtained by using methods described, e.g., in Harlow and Lane “Antibodies, A Laboratory Manual”, CSH Press, Cold Spring Harbor, 1988. Monoclonal antibodies can be prepared by the techniques originally described in Kohler and Milstein, Nature. 1975. 256: 495; and Galfré, Meth. Enzymol. 1981, 73: 3, which comprise the fusion of mouse myeloma cells to spleen cells derived from immunized mammals. Preferably, the antibody is an antibody as specified above or a polypeptide derivative thereof; more preferably, the antibody is an antibody as specified above.

As used herein, the term “aptamer” relates to a polynucleotide or polypeptide binding specifically to a target molecule by virtue of its three-dimensional structure. Preferably, the aptamer is a peptide aptamer, a “peptide aptamer” preferably being a peptide specifically interacting with P2RY2 and, thereby, inhibiting P2RY2 activity as specified herein above. Peptide aptamers, preferably, are peptides comprising 8-80 amino acids, more preferably 10-50 amino acids, and most preferably 15-30 amino acids. They can e.g. be isolated from randomized peptide expression libraries in a suitable host system like baker's yeast (see, for example, Klevenz et al., Cell Mol Life Sci. 2002, 59: 1993-1998). A peptide aptamer, preferably, is a free peptide; it is, however, also contemplated that a peptide aptamer is fused to a polypeptide serving as “scaffold”, meaning that the covalent linking to said polypeptide serves to fix the three-dimensional structure of said peptide aptamer to a specific conformation. More preferably, the peptide aptamer is fused to a transport signal, in particular a cell-penetrating peptide. Preferably, the aptamer is an aptamer as specified above or a polypeptide or polynucleotide derivative thereof, more preferably, the aptamer is an aptamer as specified above.

As used herein, the term “anticalin” relates to an artificial polypeptide derived from a lipocalin specifically binding P2RY2 and inhibiting P2RY2 activity. Similarly, a “Designed Ankyrin Repeat Protein” or “DARPin”, as used herein, is an artificial polypeptide, comprising several ankyrin repeat motifs, specifically binding P2RY2 and inhibiting P2RY2 activity. Preferably, the anticalin or DARPin is an anticalin or DARPin as specified above or a polypeptide derivative thereof, more preferably, the anticalin or DARPin is an anticalin or DARPin as specified above.

The term “non-polypeptide non-polynucleotide inhibitor macromolecule”, as used herein, relates to any and all molecules having the property of being a direct inhibitor of P2RY2 and being a macromolecule, preferably a biological macromolecule, the term “macromolecule”, as used herein, relating to molecules having a molecular mass of more than 1 kDa, preferably more than 1.5 kDa, more preferably more than 2 kDa, most preferably more than 2.5 kDa. As is understood by the skilled person, the non-polypeptide non-polynucleotide inhibitor macromolecule preferably is not a polypeptide and is not a polynucleotide, although the non-polypeptide non-polynucleotide inhibitor macromolecule may comprise substructures belonging to one of the aforesaid molecule classes. Preferably the non-polypeptide non-polynucleotide inhibitor macromolecule is a polysaccharide and/or a lipid.

Also preferably, the P2RY2 activity decreasing compound is a compound not directly inhibiting P2RY2 activity, but still reducing, preferably significantly, P2RY2 activity in a target cell. Preferably, the indirect P2RY2 activity decreasing compound is a compound decreasing the amount of P2RY2 in a target cell. Preferably, the indirect P2RY2 activity decreasing compound specifically binds to a P2RY2 encoding polynucleotide, preferably thereby significantly reducing, more preferably preventing, P2RY2 expression. Also preferably, the indirect P2RY2 activity decreasing compound is or binds to, preferably specifically binds to, a transcriptional regulator of the P2RY2 gene, preferably thereby significantly reducing, more preferably preventing, P2RY2 transcription. Thus, the indirect P2RY2 activity decreasing compound may be a transcriptional repressor of P2RY2 transcription, or may be an inhibitor of a transcriptional activator of P2RY2 transcription. The indirect P2RY2 activity decreasing compound may, however, also be a compound accelerating degradation of P2RY2 in a cell or a compound decreasing the concentration of a P2RY2 activator, in particular a nucleotide; thus, the indirect P2RY2 activity decreasing compound preferably is an apyrase (EC 3.6.1.5), a nucleoside-triphosphate phosphatase (EC 3.6.1.15), or a triphosphatase (EC 3.6.1.25).

Preferably, the indirect P2RY2 activity decreasing compound is a polynucleotide, more preferably a polynucleotide inhibiting expression or inducing degradation of a P2RY2 mRNA. More preferably, the indirect P2RY2 activity decreasing compound is selected from the group consisting of an shRNA, an siRNA, an miRNA agent, a ribozyme, an antisense molecule/an inhibitory oligonucleotide, and a CRISPR/Cas oligonucleotide. It is understood by the skilled person that inhibition of expression or induction of degradation of a specific RNA can be achieved in various ways. It is also understood by the skilled person that the exact embodiment of a polynucleotide being an indirect P2RY2 activity decreasing compound of the present invention will depend on the treatment intended.

Preferably, the indirect P2RY2 activity decreasing compound is a ribozyme. The term “ribozyme” as used herein, refers to catalytic RNA molecules possessing a well-defined tertiary structure that allows for catalyzing either the hydrolysis of one of their own phosphodiester bonds (self-cleaving ribozymes), or the hydrolysis of bonds in other RNAs, but they have also been found to catalyze the aminotransferase activity of the ribosome. The ribozymes envisaged in accordance with the present invention are, preferably, those which specifically hydrolyze their target RNAs, preferably P2RY2 mRNA, i.e., preferably RNA transcribed from a P2RY2 gene. In particular, hammerhead ribozymes are preferred in accordance with the present invention. How to generate and use such ribozymes is well known in the art (see, e.g., Hean & Weinberg (2008), RNA and the Regulation of Gene Expression: A Hidden Layer of Complexity, Chapter 1. Caister Academic Press).

More preferably, the indirect P2RY2 activity decreasing compound is an antisense oligonucleotide. The term “antisense oligonucleotide” is known to the skilled person and relates to an oligonucleotide hybridizing to a target RNA, causing the formation of a DNA/RNA hybrid. Said DNA/RNA hybrid is a substrate for RNase H, which degrades the RNA portion of said DNA/RNA hybrid. Thus, the antisense oligonucleotide comprises at least five, preferably at least seven, more preferably at least nine, most preferably at least ten, DNA nucleotides. Preferably, the antisense oligonucleotide has a length of at least 15 nucleotides, preferably at least 18 nucleotides, still more preferably at least 20 nucleotides, preferably complementary to a P2RY2 mRNA sequence as specified herein above.

Most preferably, the indirect P2RY2 activity decreasing compound is a polynucleotide inducing RNA interference. As used herein, “RNA interference (RNAi)” refers to sequence-specific, post-transcriptional gene silencing of a selected target gene by degradation of RNA transcribed from the target gene (target RNA). Target RNAs, preferably, are P2RY2 mRNAs, i.e. RNAs transcribed from a P2RY2 gene as specified herein above. It is to be understood that silencing as used herein does not necessarily mean the complete abolishment of expression. RNAi, preferably, reduces expression by at least 40%, more preferably at least 60%, even more preferably at least 80%, most preferably at least 90% as compared to the expression level in a reference without RNAi. RNAi requires in the target cell the presence of dsRNAs that are homologous in sequence to the target RNAs. The term “dsRNA” refers to RNA having a duplex structure comprising two complementary and anti-parallel nucleic acid strands. The RNA strands forming the dsRNA may have the same or a different number of nucleotides, whereby one of the strands of the dsRNA can be the target RNA. It is, however, also contemplated by the present invention that the dsRNA is formed between two sequence stretches on the same RNA molecule, e.g. by formation of a stem-loop structure. RNAi may be used to specifically inhibit expression of the target RNAs of the present invention in vivo. Accordingly, it may be used for the medical uses as specified elsewhere herein. For such therapeutic approaches, expression constructs for siRNA may be introduced into target cells of the host. Accordingly, siRNA may be combined efficiently with other therapy approaches. Methods relating to the use of RNAi to silence genes in animals, including mammals, are known in the art.

Thus, the indirect P2RY2 activity decreasing compound, preferably is an RNAi agent. As used herein, the term “RNAi agent” refers to an shRNA, a siRNA agent, or a miRNA agent as specified below. The RNAi agent of the present invention is of sufficient length and complementarity to stably interact with the target RNA, i.e. it comprises at least 15, at least 17, at least 19, at least 21, at least 22 nucleotides complementary to the target RNA. By “stably interact” is meant interaction of the RNAi agent or its products produced by the target cell with a target RNA, e.g., by forming hydrogen bonds with complementary nucleotides in the target RNA under physiological conditions.

The term “siRNA agent” as meant herein encompasses: a) a dsRNA consisting of at least 15, at least 17, at least 19, at least 21 consecutive nucleotides base-paired, i.e. forming hydrogen bonds with complementary nucleotides. b) a small interfering RNA (siRNA) molecule or a molecule comprising an siRNA molecule. The siRNA is a single-stranded RNA molecule with a length, preferably, greater than or equal to 15 nucleotides and, preferably, a length of 15 to 49 nucleotides, more preferably 17 to 30 nucleotides, and most preferably 17 to 30 nucleotides, preferably 17, 18, 19, 20, 21, 22, 23, 24, 25, 26, 27, 28, 29, or 30 nucleotides. According to the present invention, the term “molecule comprising an siRNA molecule” includes RNA molecules from which an siRNA is processed by a cell, preferably by a mammalian cell. Thus, a molecule comprising an siRNA molecule, preferably, is a small hairpin RNA, also known as shRNA. As used herein, the term “shRNA” relates to a, preferably artificial, RNA molecule forming a stem-loop structure comprising at least 10, preferably at least 15, more preferably at least 17, most preferably at least 20 nucleotides base-paired to a complementary sequence on the same mRNA molecule (“stem”), i.e. as a dsRNA, separated by a stretch of non-base-paired nucleotides (“loop”). c) a polynucleotide encoding a) or b), wherein, preferably, said polynucleotide is operatively linked to an expression control sequence. Thus, the function of the siRNA agent to inhibit expression of the target gene can be modulated by said expression control sequence. Preferred expression control sequences are those, which can be regulated by exogenous stimuli, e.g. the tet operator, whose activity can be regulated by tetracycline, or heat inducible promoters. Alternatively or in addition, one or more expression control sequences can be used which allow tissue-specific expression of the siRNA agent.

It is, however, also contemplated by the current invention that the RNAi agent is a miRNA agent. A “miRNA agent” as meant herein encompasses: a) a pre-microRNA, i.e. an mRNA comprising at least 30, at least 40, at least 50, at least 60, at least 70 nucleotides base-paired to a complementary sequence on the same mRNA molecule (“stem”), i.e. as a dsRNA, separated by a stretch of non-base-paired nucleotides (“loop”). b) a pre-microRNA, i.e. a dsRNA molecule comprising a stretch of at least 19, at least 20, at least 21, at least 22, at least 23, at least 24, at least 25 base-paired nucleotides formed by nucleotides of the same RNA molecule (stem), separated by a loop. c) a microRNA (miRNA), i.e. a dsRNA comprising at least 15, at least 17, at least 18, at least 19, at least 21 nucleotides on two separate RNA strands. d) a polynucleotide encoding a) or b), wherein, preferably, said polynucleotide is operatively linked to an expression control sequence as specified above.

Also preferably, the indirect P2RY2 activity decreasing compound comprises at least one, preferably two, CRISPR/Cas oligonucleotides. The CRISPR/Cas system has been known for several years as a convenient system for inducing knock-out mutations, i.e. deletions, preferably of chromosomal genes. The skilled person knows how to design appropriate oligonucleotides, which are, preferably, expressed from a vector, to induce deletion of a DNA sequence of interest. Preferably, said deletion is a partial deletion, more preferably deletion of a portion of the gene essential for function; most preferably said deletion is a complete deletion of at least the whole coding region. As is known in the art, single CRISPR/Cas oligonucleotides can be used to introduce short insertions, deletions, and/or frameshifts in a coding sequence of interest, while two CRISPR/Cas oligonucleotides may be used to mediate larger deletions or deletions of exons, coding regions and/or whole genes. As referred to herein, two CRISPR/Cas oligonucleotides together mediating such partial or complete deletion of at least one exon, coding region and/or whole gene are referred to as a “pair of CRISPR/Cas oligonucleotides”.

Also preferably, the indirect P2RY2 activity decreasing compound is a polypeptide comprising a lysosome-degradation sequence, preferably a chaperone-mediated autophagy-targeting motif (CTM). Preferably, said CTM-comprising polypeptide specifically binds to P2RY2; e.g. the CTM-comprising polypeptide may further comprise an antibody specifically binding to P2RY2. As the skilled person will understand, the CTM-conjugated antibody does not necessarily have to be an inhibitory P2RY2 antibody as specified herein above; it is, however, preferred that the antibody is a P2RY2-specific antibody. Preferably, the CTM-comprising polypeptide specifically binding to P2RY2 binds to P2RY2 with a KD value at least 10 fold, preferably at least 20 fold, more preferably at least 50 fold, most preferably at least 100 fold higher than the KD value of said CTM-comprising polypeptide for other G-protein coupled receptors. More preferably, the CTM-comprising polypeptide specifically binding to P2RY2 does not detectably bind to other cellular proteins, preferably including non-P2RY2 G-protein coupled receptors. As will be understood by the skilled person, in case the indirect P2RY2 activity decreasing compound is a CTM-comprising polypeptide, said CTM-comprising polypeptide does not have to be, but may be, a direct P2RY2 activity decreasing compound. Thus, preferably, the CTM-comprising polypeptide also is a direct P2RY2 activity decreasing compound.

Also preferably, the activity modulator is an activity increasing compound; thus, preferably the P2RY2 activity modulator is a P2RY2 activity increasing compound, preferably a P2RY2-specific activity increasing compound. Preferably, the P2RY2-specific activity increasing compound activates non-P2RY2-specific activity by at most 50%, preferably at most 25%, more preferably by at most 10%, at a concentration activating P2RY2 activity by 90%. The activity of a P2RY2 activity increasing compound is, preferably, determined in vitro by assaying the amount and/or enzymatic activity of P2RY2 as specified elsewhere herein, preferably as shown herein in the Examples. Also preferably, said P2RY2 activity increasing compound decreases T cell-mediated autoimmune activity in vitro and/or in vivo. Compounds increasing activity of a known polypeptide gene product such as P2RY2 can be provided by the skilled person by standard methods of molecular biology, e.g. as specified herein below. Preferably, the P2RY2 activity modulator is a P2RY2 activity increasing compound in case the T cell immunotherapy is therapy of T cell mediated autoimmune disease as specified herein below.

Preferably, the P2RY2 activity increasing compound is a direct P2RY2 activity increasing compound binding to and activating activity of P2RY2, more preferably is a small molecule activator, an activator polypeptide, an activator polynucleotide, or a non-polypeptide non-polynucleotide activator macromolecule.

The term “small molecule” has been specified herein above. In accordance, the term “small molecule activator”, as used herein, relates to a small molecule compound increasing P2RY2 activity. Small molecule activators of P2RY2 are known in the art and include in particular nucleotides and derivatives thereof. Thus, the small molecule activator of P2RY2 preferably is ATP or UTP or a derivative thereof, more preferably is MRS 2768 (Uridine-5′-tetraphosphate δ-phenyl ester, CAS No: 1047980-83-5), Uridine-5′-(γ-thio)-triphosphate (CAS No: 1266569-94-1), 4-Thiouridine-5′-O-(β,γ-difluoromethylene)triphosphate (CAS No: 1657025-60-9), Denufosol (CAS No: 211448-85-0), or Diquafosol (CAS No: 59985-21-6).

The term “activator polypeptide”, as used herein, includes any and all polypeptides having an activating effect on P2RY2. Preferably, the activator polypeptide is an activator antibody, preferably a P2RY2 agonist antibody.

Also preferably, the P2RY2 activity increasing compound is an indirect P2RY2 activity increasing compound, i.e. a compound not binding to P2RY2 but nonetheless increasing P2RY2 activity. Preferably, the indirect P2RY2 activator is (i) a polypeptide comprising a P2RY2 polypeptide; (ii) a polynucleotide encoding a polypeptide comprising a P2RY2 polypeptide; (iii) a vector comprising the polynucleotide of (ii); (iv) a host cell comprising the polynucleotide of (ii) and/or the vector of (iii); or (v) any combination of (i) to (iv). The indirect P2RY2 activity increasing compound may, however, also be an apyrase inhibitor, a nucleoside-triphosphate phosphatase inhibitor, or a triphosphatase inhibitor.

Exemplary sequences of P2RY2 polypeptides and of P2RY2 encoding polynucleotides have been indicated herein above. Exemplary vectors suitable for comprising a polynucleotide encoding a polypeptide comprising a P2RY2 polypeptide, in particular expression vectors, have been discussed herein above as well.

The term “T cell” is understood by the skilled person to relate to a lymphocyte expressing at least one type of T cell receptor. Preferably, the T cell is a CD8+ T cell recognizing MHC class I molecules on the surface of target cells, or is a CD4+ T cell recognizing MHC class II molecules on the surface of target cells, more preferably is a CD8+ T cell. Preferably, the T cell is a cytotoxic T cell, more preferably a CD8+ cytotoxic T cell, which may also be referred to as “killer cell”. Also preferably, the T cell is a regulatory or helper T cell, more preferably a regulatory T cell. Preferably, the T cell is reactive to cancer cells, i.e. is a cancer-reactive T cell or is reactive to cells presenting a T cell autoantigen, i.e. is a T cell mediating autoimmune disease. Thus, preferably, the T cell expresses a TCR recognizing a cancer antigen, preferably a cancer-specific antigen. Also preferably, the T cell expresses a TCR recognizing an autoimmune T cell antigen, preferably a specific autoimmune T cell antigen. Preferably, the T cell is a recombinant cell expressing a chimeric antigen receptor (CAR) and/or a recombinant T cell receptor. Methods of producing appropriate recombinant T cells are known in the art.

The terms “treating” and “treatment” refer to an amelioration of a disease or disorder referred to herein or the symptoms accompanied therewith to a significant extent; as used herein, the term includes prevention of deterioration of a disease, disorder, or symptoms associated therewith. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating comprises activating an immune response against an agent causing or mediating disease, i.e. against cancer cells; or comprises inhibiting an immune response against host cells causing or mediating disease in case of an autoimmune disease. Preferably, treating cancer is reducing tumor and/or cancer cell burden in a subject. As will be understood by the skilled person, effectiveness of treatment of e.g. cancer is dependent on a variety of factors including, e.g. cancer stage and cancer type. Also preferably, cancer treatment further comprises at least one of surgery, chemotherapy, and radiotherapy.

The terms “preventing” and “prevention” refer to retaining health with respect to the diseases or disorders referred to herein for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification. In the context of cancer treatment, preventing in particular relates to preventing cancer development, preventing metastasis formation, and/or preventing relapse, preferably relates to preventing metastasis formation and/or preventing relapse.

The term “immunotherapy”, as used herein, relates to the treatment and/or prevention of disease, preferably cancer and/or autoimmune disease, by modulation of the immune response of a subject. Said modulation may be inducing, enhancing, or suppressing said immune response, e.g. by administration of a P2RY2 modulator and, optionally, additionally of at least one immune checkpoint modulator and/or cytokine, preferably an immune checkpoint inhibitor and/or a cytokine. Preferably, the cytokine is an interferon, an interleukin, or a chemokine in such case. The immunotherapy may also further comprise administration of at least one T cell engager, i.e. a molecule tethering a T cell to a target cell; corresponding T cell engagers, e.g. bispecific T cell engagers (BiTEs) such as bispecific antibodies, are known in the art.

The term “T cell immunotherapy” relates to a therapy of disease comprising modulation of T cell activity, e.g. of cancer-specific T cells (cancer T cell immunotherapy); as used herein, the term modulation of T cell activity relates to a modulation of the activity of T cells, preferably in a subject, which may entail modulation of the activity of T cells already present, e.g. in a subject, but may also comprise administration of T cells enriched and/or genetically modified to recognize a target of interest, preferably a cancer antigen. Thus, the T cell immunotherapy may in particular be an adoptive T cell immunotherapy, preferably comprising administration of tumor-infiltrating lymphocytes (TIL), i.e. be TIL therapy, and/or comprising administration of genetically engineered T cells, in particular genetically modified T cells expressing a chimeric antigen receptor (CAR) and/or a recombinant T cell receptor (TCR), i.e. may be an engineered T cell immunotherapy. As is understood by the skilled person from the disclosure herein, as specified herein, T cells are effector cells mediating or contributing to T cell immunotherapy; however, said T cells do not have to necessarily be the target cells of the P2RY2 activity modulator; thus, the effect of the P2RY2 activity modulator may preferably be mediated via an effect of the P2RY2 activity modulator on target cells of said T cells, e.g. cancer cells and/or cells presenting an autoimmune T cell antigen.

Preferably, T cell immunotherapy further comprises at least one, preferably at least two, more preferably all three, of administration of an immune checkpoint inhibitor, administration of a chemokine, and identification of said subject as amenable to T cell immunotherapy before treatment. More preferably, T cell immunotherapy comprises identification of said subject as amenable to T cell immunotherapy before treatment, or comprises identification of said subject as amenable to T cell immunotherapy before treatment. and (i) administration of an immune checkpoint inhibitor, (ii) administration of a cytokine, and/or (iii) administration of a T cell engager. However, T cell immunotherapy may also comprise in situ activation of a subject's existing T cells, thereby making administration of T cells dispensable. Also, in particular in the case of cancer therapy, T cell immunotherapy may comprise isolation of T cells, activation of said T cells in vitro, and their re-administration to the same or a different subject, preferably to the same subject (autologous treatment).

Preferably, T cell immunotherapy is, typically, administered systemically, preferably orally or parenterally, e.g. by intravenous administration, or is administered topically, preferably intra-tumorally, topically on a body surface, or by inhalation; in case of cancer treatment, topical administration may be intratumoral or peritumoral, and/or topical at a site of tumor excision. Administration may, however, also be into a blood vessel, typically an artery, afferent to an intended site of effect, such as a tumor. However, depending on the nature of the formulation and the desired therapeutic application, the medicament may be administered by other routes as well. T cell immunotherapy is preferably administered in conventional dosage forms prepared by combining the drug with standard pharmaceutical carriers according to conventional procedures. These procedures may involve mixing, granulating, and compression, or dissolving the ingredients as appropriate to obtain the desired preparation.

The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body. Preferably, also included by the term cancer is a recurrence of a cancer. Thus, preferably, the cancer is a solid cancer, a metastasis, or a relapse thereof. Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, fibrosarcoma, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sezary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenstrom macroglobulinemia, and wilms tumor. More preferably, the cancer is a solid cancer, a metastasis, or a relapse thereof. More preferably, said cancer is glioblastoma, pancreatic ductal adenocarcinoma, osteosarcoma, or a brain metastasis of a non-brain primary tumor.

The term “cancer antigen” relates to an antigen, preferably a polypeptide, expressed by a cancer cell. Preferably, the cancer antigen is expressed at an at least 5 fold, preferably at least 10 fold, more preferably at least 25 fold, lower rate in non-cancer cells. Preferably, the cancer antigen is not expressed in non-tumor cells of the same tissue in a subject, more preferably is not expressed in non-cancer cells of a subject; thus, the cancer antigen preferably is a cancer-specific antigen. More preferably, the cancer antigen is a neoantigen and/or comprises a neoepitope, expressed by cancer cells. The cancer antigen may, however, also be an antigen foreign to the subject, in particular a viral, bacterial, or microbial antigen, preferably cancer antigen. In particular viral cancer antigens are known in the art and include e.g. antigens derived from the human papillomavirus E6 and E7 genes, the Epstein-Barr virus LMP-1 protein, and the like. Preferably, one or more peptides of the cancer antigen are presented via MHC molecules, more preferably MHC class-I molecules, on the surface of host cells producing said cancer antigen as “cancer epitopes”, which preferably are cancer-specific epitopes or, as specified above, cancer-associated viral epitopes and/or cancer neoepitopes.

The term “autoimmune T cell activating antigen” is, in principle, known to the skilled person to relate to any antigen presented by a cell of a subject, the recognition of which causes, aggravates, or contributes to autoimmune disease, preferably T cell mediated autoimmune disease. T cell mediated autoimmune diseases are known in the art; preferably, the T cell mediated autoimmune disease is selected from the list consisting of multiple sclerosis, celiac disease, rheumatoid arthritis, type 1 diabetes mellitus, hypothyroidism, and Addison's disease.

The definitions made above apply mutatis mutandis to the following. Additional definitions and explanations made further below also apply for all embodiments described in this specification mutatis mutandis.

Advantageously, it was found in the work underlying the present invention that P2RY2 is both a modulator of B7-H3 expression and an inhibitor of activation of T cells, e.g. against cancer cells. In accordance, inhibition of P2RY2 was found to improve activity of T cells against cancer cells both in an B7-H3 dependent and a B7-H3 independent manner. Thus, inactivation of P2RY2 was found to remove two T cell inhibition pathways, improving efficacy of T cell immunotherapy.

The present invention also relates to a pharmaceutical composition comprising the P2RY2 activity modulator of the present invention for use in T cell immunotherapy.

The terms “medicament” and “pharmaceutical composition” are used essentially interchangeably herein and are, in principle, known to the skilled person. As referred to herein, the terms relate to any composition of matter comprising the specified active agent(s) as pharmaceutically active compound(s) and one or more excipient. The pharmaceutically active compound(s) can be present in liquid or dry, e.g. lyophilized, form. It will be appreciated that the form and character of the pharmaceutical acceptable excipient, e.g. carrier or diluent, is dictated by the amount of active ingredient with which it is to be combined, the route of administration, and other well-known variables. The excipient(s) must be acceptable in the sense of being compatible with the other ingredients of the formulation and being not deleterious to the recipient thereof. The excipient employed may include a solid, a gel, or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, physiological saline, Ringer's solutions, dextrose solution, and Hank's solution, syrup, oil, water, emulsions, various types of wetting agents, and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pennsylvania. The excipient(s) is/are selected so as not to affect the biological activity of the combination. The excipient may, however, also be selected to improve uptake of the active agent into a host cell, in particular a target cell. Thus, the excipient may also be a viral particle and/or a lipid vesicle, preferably a viral particle and/or a lipid vesicle known to mediate entry and/or fuse with the target cell of interest.

The medicament is, preferably, administered by a route as specified herein above. A therapeutically effective dose refers to an amount of the effector polypeptide or expression construct encoding the same to be used in a medicament which prevents, ameliorates or cures the symptoms accompanying a disease or condition referred to in this specification. Therapeutic efficacy and toxicity of a drug can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and by clinical factors. As is well known in the medical arts, dosages for any one patient depends upon many factors, including the patient's size, age, the particular formulation of the medicament to be administered, sex, time and route of administration, general health, and other drugs being administered concurrently. The medicament referred to herein is, preferably, administered at least once, e.g. as a bolus. However, the medicament may be administered more than one time and, preferably, at least twice, e.g. permanently or periodically after defined time windows. Progress can be monitored by periodic assessment. Dosage recommendations may be indicated in the prescriber or user instructions in order to anticipate dose adjustments depending on the considered recipient.

The medicament according to the present invention may comprise further active agents in addition to the aforementioned active agent(s). Preferably, the pharmaceutically active compound according to the invention is to be applied together with at least one further drug and, thus, may be formulated together with this at least one further drug as a medicament. More preferably, in case of cancer treatment, said at least one further active agent is a chemotherapeutic agent or a further immunotherapeutic agent, such as an immune checkpoint modulator; in case of autoimmune disease treatment, said at least one further active agent preferably is an anti-inflammatory agent. Also, it is to be understood that the formulation of a pharmaceutical composition preferably takes place under GMP standardized conditions or the like in order to ensure quality, pharmaceutical safety, and effectiveness of the medicament.

The present invention further relates to a use of the P2RY2 activity modulator as specified herein above in the manufacture of a pharmaceutical composition for T cell immunotherapy.

The present invention also relates to a polynucleotide encoding a P2RY2 activity modulator of the present invention for use in T cell immunotherapy. As will be appreciated by the skilled person, the P2RY2 activity modulator encoded by the polynucleotide preferably is a polypeptide modulator or a polynucleotide modulator.

The present invention also relates to a vector comprising the polynucleotide encoding a P2RY2 activity modulator of the present invention for use in T cell immunotherapy. Suitable vectors have been specified herein above, expression vectors, in particular mammalian expression vectors being preferred.

The present invention also relates to a host cell comprising the P2RY2 activity modulator, the polynucleotide, and/or the vector of the present invention, for use in T cell immunotherapy.

As used herein, the term “host cell” relates to any cell capable of receiving, and preferably maintaining, the polynucleotide, the vector, and/or the P2RY2 activity modulator, as specified herein above. Preferably, the host cell is capable of expressing a P2RY2 activity modulator encoded on the expression polynucleotide and/or expression vector as specified herein above. Also preferably, the host cell is a cell capable of providing a P2RY2 activity modulator, preferably to a target cell as specified herein below, e.g. by cell fusion, secretion, or the like. Thus, the host cell may also be a cell producing viral particles and/or liposomes comprising the P2RY2 activity modulator as specified herein. Preferably, the host cell is a eukaryotic cell, preferably an animal cell, e.g. an insect cell or a mammalian cell. More preferably, the host cell is a cell of a livestock, companion, or laboratory animal. Most preferably, the host cell is a human cell. Preferred host cells for determining P2RY2 regulation of B7-H3 expression are cell lines THP-1, U937, KBM7, and K562; preferred host cells for determining P2RY2 activity are cell lines BxPC3, MCF7 and the aforesaid host cells for determining P2RY2 regulation of B7-H3; the aforesaid cell lines are available from generally known commercially sources and strain collections.

Preferably, the host cell is a target cell, the term “target cell”, as used herein, relating to a host cell in which P2RY2 activity modulation is desirable. The target cell may be of any cell type causing or contributing to disease, in which case it may also be referred to as “disease-mediating cell”; thus, a target cell preferably is an immune cell, preferably a T cell, a monocyte, or a tissue macrophage. More preferably, the target cell is a cancer cell and/or a cell presenting an autoimmune T cell antigen.

The present invention also relates to a method of identifying a subject amenable to T cell immunotherapy comprising
(A) determining in a sample of said subject the activity of P2RY2;
(B) comparing the activity determined in step (B) to a reference; and
(C) identifying a subject amenable to T cell immunotherapy based on the comparison of step (B).

The method of the present invention, preferably, is an in vitro method. Moreover, it may comprise steps in addition to those explicitly mentioned above. For example, further steps may relate, e.g., to obtaining a sample for step a), or determining the activity of P2RY2 in a reference in step b). Moreover, one or more of said steps may be assisted or performed by automated equipment.

The term “determining”, as used herein, refers to quantitatively or semiquantitatively determining an analyte by measuring at least one characteristic feature of the analyte to be determined in a sample. Characteristic features in accordance with the present invention are features which characterize the physical and/or chemical properties including biochemical properties of an analyte. Such properties include, e.g., molecular weight, as e.g. determined by mass spectrometry; nucleic acid sequence, as e.g. determined by sequencing; capability to react with other compounds, in particular hybridization to one or more probe oligonucleotide or primer oligonucleotide(s), or activation of a downstream component in a signaling pathway, e.g. as specified herein below; capability to elicit a response in a biological read out system (e.g., induction of a reporter gene); and the like. Values for the aforesaid properties may serve as characteristic features and can be determined by techniques well known in the art. Moreover, the value determined may be any value which is derived from the values of the physical and/or chemical properties of an analyte by standard operations, e.g., mathematical calculations such as multiplication, division or logarithmic calculus. Preferably, the value determined for a is a normalized value, e.g. normalized to a control analyte.

The term “activity of P2RY2” relates to the activity of the P2RY2 polypeptide in a cell, preferably signal transduction, in particular G-protein receptor signaling. In accordance, activity of P2RY2 may be determined by determining activity and/or amount of P2RY2 in a cell, but may also be determined by determining the activity and/or amount of activated signaling molecules downstream of P2RY2, in particular, e.g. products of the phosphatidylinositol-calcium second messenger system, in particular intracellular calcium concentration. Preferably, determining the activity of P2RY2 is determining the amount of P2RY2.

The method of identifying a subject amenable to T cell immunotherapy may comprise additional determining activity of B7-H3 (B7 homolog 3, also referred to as CD276). Human B7-H3 preferably has the amino acid sequence of Genbank Acc. No. NP_001019907.1 or a variant thereof, and is preferably encoded by a polynucleotide comprising the nucleic acid sequence of Genbank Acc. No. NM_001024736.2 or a variant thereof. The activity of B7-H3 may be determined by determining its immune checkpoint activity and/or by determining the amount of B7-H3 in a cell, preferably is determined by determining the amount of B7-H3. As referred to herein, the method of identifying a subject amenable to T cell immunotherapy does not comprise determining B7-H3 alone.

The term “comparing”, as used herein, encompasses comparing the activity which is comprised by a sample with an activity in a suitable reference as specified elsewhere herein. Comparing may also comprise calculating a score. It is to be understood that comparing as referred to herein refers to a comparison of corresponding parameters or values, e.g., an absolute amount of a polypeptide is compared to an absolute reference amount; a concentration of a polypeptide is compared to a reference concentration; or an activity is compared to the same type of activity in a reference, a score is compared to a reference score, and the like. The comparison referred to in the methods of the present invention may be carried out manually or computer assisted. For a computer assisted comparison, the value determined may be compared to a value corresponding to a suitable reference which is stored in a database by a computer program. The computer program may further evaluate the result of the comparison by means of an expert system. Accordingly, the result of the identification referred to herein may be automatically provided in a suitable output format.

The terms “reference”, “reference value”, “reference amount”, or “reference score”, as used herein, refer to an activity, amount and/or a value derived therefrom which allows assessing if the subject from which the sample is derived is amenable to T cell immunotherapy, or not. A suitable reference may be determined from a reference subject, preferably a population of reference subjects, preceding, simultaneously, or subsequently, with the sample. Preferably, the reference is a pre-determined reference which may be stored e.g. in a database. Also preferably, the reference is a fixed value or range of values for all subjects to be tested.

References can, in principle, be calculated for a group or cohort of subjects as specified herein based on the average or median values for a given analyte by applying standard methods of statistics. In particular, accuracy of a test such as a method aiming to predict an event, or not, is best described by its receiver-operating characteristics (ROC) (see especially Zweig (1993), Clin. Chem. 39:561). Preferably, the reference amount as used herein is derived from samples of a population of subjects for which it is known if their donors were amendable to T cell immunotherapy. This reference may be a discrete figure or may be a range of figures. A reference may, preferably, be derived from a subject or group of subjects being known to have been amenable to T cell immunotherapy; in such case, preferably, a P2RY2 activity being essentially equal to or decreased compared to said reference is indicative of a subject amenable to T cell immunotherapy, while an activity increased compared to said reference is indicative of a subject not amenable to T cell immunotherapy. A reference may, preferably, also be derived from a subject or group of subjects known to later have not been amenable to T cell immunotherapy; in such case, preferably, a P2RY2 activity being essentially equal to or increased compared to said reference is indicative of a subject not amenable to T cell immunotherapy, while an activity decreased compared to said reference is indicative of a subject amenable to T cell immunotherapy. It is to be understood that the aforementioned activities may vary due to statistics and errors of measurement. A deviation, i.e. a decrease or an increase of the activity referred to herein is, preferably, a statistically significant deviation, i.e. a statistically significant decrease or a statistically significant increase. On the other hand, the number of samples, in particular from the subject under evaluation, so statistical evaluation may not be possible or may be deemed dispensable.

The term “subject”, as referred to herein, relates to a vertebrate animal, preferably a mammal, in particular a livestock, companion, or laboratory animal. Most preferably, subject is a human. Preferably, the subject has been diagnosed to suffer from cancer or autoimmune disease as specified herein above.

The term “sample” refers to a sample of a bodily fluid, preferably a sample of blood, plasma, serum, saliva, sputum, urine, or a sample comprising separated cells or to a sample from a tissue or an organ. more preferably is a cancer sample, in particular a tumor or metastasis sample, which may be obtained e.g. by biopsy. Separated cells may be obtained from the body fluids, such as lymph, blood, plasma, serum, liquor and other, or from the tissues or organs by separating techniques such as centrifugation or cell sorting. Preferably, the sample is a tissue or body fluid sample, more preferably the sample is a sample of a body fluid, preferably a blood sample, serum, or plasma sample, most preferably a serum sample. More preferably, the sample is a sample known or suspected to comprise cancer cells The sample can be obtained from the subject by routine techniques which are well known to the person skilled in the art, e.g., venous or arterial puncture or open biopsy including aspiration of tissue or cellular material from a subject. For those areas which cannot be easily reached via an open biopsy, a surgery and, preferably, minimal invasive surgery can be performed.

The present invention also relates to a method for treating a subject suffering from an immune-aggravated disease, said method comprising
(i) contacting said subject with a P2RY2 activity modulator; and
(ii) thereby treating immune-aggravated disease.

The term “immune aggravated disease”, as used herein, includes any and all diseases in which the immune response contributes to severity of disease, wherein the contribution of the immune response may be an immune response which is decreased compared to a reference, e.g. in cancer, or may be an immune response which is increased compared to a reference, e.g. in autoimmune disease. In accordance, the skilled person will select an appropriate P2RY2 activity modulator to achieve the desired effect in view of the description herein.

The present invention also relates to a method for identifying a P2RY2 activity modulator, said method comprising
(I) contacting a host cell with a candidate compound suspected to be a P2RY2 activity modulator;
(II) determining B7-H3 activity in said host cell;
(III) comparing the B7-H3 activity determined in step (II) to a control; and
(IV) identifying a P2RY2 activity modulator based on the comparison in step (III).

The term “B7-H3 activity” relates to the activity of the B7-H3 polypeptide in a cell, preferably signal transduction. In accordance, activity of B7-H3 may be determined by determining activity and/or amount of B7-H3 in a cell, but may also be determined by determining the activity and/or amount of activated signaling molecules downstream of B7-H3, as specified elsewhere herein. Preferably, determining B7-H3 activity is determining the amount of B7-H3.

The term “control” is understood by the skilled person; preferably, a control is a determination of B7-H3 in a host cell not treated with a candidate compound suspected to be a P2RY2 activity modulator, but, preferably, treated identically otherwise. As is understood by the skilled person, a P2RY2 activity modulator is identified in case the B7-H3 activity determined in the host cell treated with a candidate compound suspected to be a P2RY2 activity modulator is different from the B7-H3 activity determined in the untreated (control) host cell.

The present invention also relates to a kit comprising a P2RY2 activity modulator and (i) means for determining P2RY2 activity and/or B7-H3 activity in a sample, (ii) a B7-H3 activity modulator.

The term “kit”, as used herein, refers to a collection of the aforementioned compounds, means or reagents which may or may not be packaged together. The components of the kit may be comprised by separate vials (i.e. as a kit of separate parts) or provided in a single vial, e.g. as a composition as specified herein above. The housing of the kit in an embodiment allows translocation of the compounds of the kit, in particular common translocation; thus, the housing may in particular be a transportable container comprising all specified components. Moreover, it is to be understood that the kit of the present invention may be used for practicing the methods referred to herein above. It is, in an embodiment, envisaged that all components are provided in a ready-to-use manner for practicing the methods referred to above. Further, the kit preferably contains instructions for carrying out said methods. The instructions can be provided by a user's manual on paper or in electronic form. For example, the manual may comprise instructions for interpreting the results obtained when carrying out the aforementioned methods using the kit.

Preferably, the kit comprises further compounds, such as a reaction buffer, a hybridization solution, a lysis buffer, and the like. Preferably, the kit is adapted for use in a method of the present invention, more preferably is adapted to comprise all reagents required to perform said method or methods.

In view of the above, the following embodiments are particularly envisaged:

Embodiment 1: A P2Y purinoceptor 2 (P2RY2) activity modulator for use in T cell immunotherapy.

Embodiment 2: The P2RY2 activity modulator for use of embodiment 1, wherein said T cell immunotherapy is an adoptive T cell immunotherapy, preferably is tumor-infiltrating lymphocyte (TIL) therapy, and/or engineered T cell immunotherapy, in particular chimeric antigen receptor (CAR) and/or recombinant T cell receptor immunotherapy.

Embodiment 3: The P2RY2 activity modulator for use of embodiment 1 or 2, wherein said P2RY2 modulator is a P2RY2 activity decreasing compound.

Embodiment 4: The P2RY2 activity modulator for use of any one of embodiments 1 to 3, wherein said T cell immunotherapy is cancer T cell immunotherapy, preferably further comprising at least one of administration of an immune checkpoint inhibitor, administration of a cytokine, administration of a T cell engager, and identification of said subject as amenable to T cell immunotherapy, preferably according to any one of embodiments 28 to 30.

Embodiment 5: The P2RY2 activity modulator for use of any one of embodiments 1 to 4, wherein said P2RY2 activity decreasing compound increases T cell anti-cancer activity in vitro and/or in vivo.

Embodiment 6: The P2RY2 activity modulator for use of any one of embodiments 1 to 5, wherein said P2RY2 activity decreasing compound is a direct P2RY2 activity decreasing compound specifically binding to and inhibiting P2RY2.

Embodiment 7: The P2RY2 activity modulator for use of any one of embodiments 1 to 6, wherein said direct P2RY2 activity decreasing compound is a small molecule inhibitor, an inhibitor polypeptide, an inhibitor polynucleotide, or a non-polypeptide non-polynucleotide inhibitor macromolecule.

Embodiment 8: The P2RY2 activity modulator for use of embodiment 7, wherein said small molecule inhibitor is AR-C 118925XX (CAS No: 216657-60-2).

Embodiment 9: The P2RY2 activity modulator for use of embodiment 7, wherein said inhibitor polypeptide is selected from the list consisting of an antibody, an aptamer, an anticalin, and a Designed Ankyrin Repeat Protein (DARPin).

Embodiment 10: The P2RY2 activity modulator for use of embodiment 7, wherein said inhibitor polynucleotide is a polynucleotide aptamer.

Embodiment 11: The P2RY2 activity modulator for use of any one of embodiments 1 to 5, wherein said P2RY2 activity decreasing compound is an indirect P2RY2 activity decreasing compound decreasing the amount of P2RY2 in a target cell.

Embodiment 12: The P2RY2 activity modulator for use of embodiment 11, wherein said indirect P2RY2 activity decreasing compound is selected from the list consisting of an shRNA, a siRNA, a miRNA agent, an antisense oligonucleotide, a ribozyme, and a CRISPR/Cas oligonucleotide, preferably a pair of CRISPR/Cas oligonucleotides.

Embodiment 13: The P2RY2 activity modulator for use of any one of embodiments 1 to 12, wherein said P2RY2 activity modulator is administered topically, preferably intra-tumorally.

Embodiment 14: The P2RY2 activity modulator for use of any one of embodiments 1 to 3, wherein said P2RY2 modulator is a P2RY2 activity increasing compound.

Embodiment 15: The P2RY2 activity modulator for use of embodiment 14, wherein said immunotherapy is immunotherapy of autoimmune disease.

Embodiment 16: The P2RY2 activity modulator for use of embodiment 14 or 15, wherein said P2RY2 activity increasing compound is a direct P2RY2 activity increasing compound binding to and increasing activity of P2RY2.

Embodiment 17: The P2RY2 activity modulator for use of any one of embodiments 14 to 16, wherein said P2RY2 activity increasing compound is a small molecule activator, an activator polypeptide, an activator polynucleotide, or a non-polypeptide non-polynucleotide activator macromolecule.

Embodiment 18: The P2RY2 activity modulator for use of embodiment 17, wherein said small molecule activator is a nucleotide or a derivative thereof, preferably is ATP or UTP or a derivative thereof, more preferably is MRS 2768 (Uridine-5′-tetraphosphate δ-phenyl ester, CAS No: 1047980-83-5), Uridine-5′-(γ-thio)-triphosphate (CAS No: 1266569-94-1), 4-Thiouridine-5′-O-(β,γ-difluoromethylene)triphosphate (CAS No: 1657025-60-9), Denufosol (CAS No: 211448-85-0), or Diquafosol (CAS No: 59985-21-6).

Embodiment 19: The P2RY2 activity modulator for use of embodiment 17, wherein said activator polypeptide is a P2RY2 agonist, preferably a P2RY2 agonist antibody.

Embodiment 20: The P2RY2 activity modulator for use of embodiment 14 or 15, wherein said P2RY2 activator is an indirect P2RY2 activator increasing the amount of P2RY2 in a target cell.

Embodiment 21: The P2RY2 activity modulator for use of embodiment 20, wherein said indirect P2RY2 activator is
(i) a polypeptide comprising a P2RY2 polypeptide;
(ii) a polynucleotide encoding a polypeptide comprising a P2RY2 polypeptide;
(iii) a vector comprising the polynucleotide of (ii);
(iv) a host cell comprising the polynucleotide of (ii) and/or the vector of (iii); or
(v) any combination of (i) to (iv).

Embodiment 22: The P2RY2 activity modulator for use of embodiment 20, wherein said vector is a virus.

Embodiment 23: A pharmaceutical composition comprising the P2RY2 activity modulator as specified in any one of embodiments 5 to 22 for use in T cell immunotherapy.

Embodiment 24: Use of the P2RY2 activity modulator as specified in any one of embodiments 5 to 22 in the manufacture of a pharmaceutical composition for T cell immunotherapy.

Embodiment 25: A polynucleotide encoding a P2RY2 activity modulator as specified in any one of embodiments 5 to 22, for use in T cell immunotherapy.

Embodiment 26: A vector comprising the polynucleotide according to embodiment 25, for use in T cell immunotherapy.

Embodiment 27: A host cell comprising the P2RY2 activity modulator as specified in any one of embodiments 5 to 22, the polynucleotide of embodiment 25, and/or the vector according to embodiment 26, for use in T cell immunotherapy.

Embodiment 28: A method of identifying a subject amenable to T cell immunotherapy comprising
(A) determining in a sample of said subject the activity of P2RY2;
(B) comparing the amount determined in step (B) to a reference; and
(C) identifying a subject amenable to T cell immunotherapy based on the comparison of step (B).

Embodiment 29: The method of embodiment 28, wherein said reference is derived from a (i) subject or group of subjects known to be amenable to immunotherapy or (ii) a subject or group of subjects known not to be amenable to immunotherapy.

Embodiment 30: The method of embodiment 28 or 29, wherein determining the activity comprises determining the amount of P2RY2 and/or of at least one of its downstream signaling molecules, preferably intracellular calcium concentration.

Embodiment 31: A method for treating a subject suffering from an immune-aggravated disease, said method comprising
(a) contacting said subject with a P2RY2 activity modulator; and
(b) thereby treating immune-aggravated disease.

Embodiment 32: The method of embodiment 31, wherein said immune-aggravated disease is cancer or autoimmune disease.

Embodiment 33: A method for identifying a P2RY2 activity modulator, said method comprising
(I) contacting a host cell with a candidate compound suspected to be a P2RY2 activity modulator;
(II) determining B7-H3 activity in said host cell;
(III) comparing the B7-H3 activity determined in step (II) to a control; and
(IV) identifying a P2RY2 activity modulator based on the comparison in step (III).

Embodiment 34: A kit comprising a P2RY2 activity modulator and (i) means for determining P2RY2 activity and/or B7-H3 activity in a sample, (ii) a B7-H3 activity modulator.

Embodiment 35: The subject matter of any one of embodiments 23 to 33, wherein said T cell immunotherapy is T cell immunotherapy comprising administration of at least one P2RY2 activity modulator and, preferably, further comprises at least one of administration of an immune checkpoint inhibitor, administration of a cytokine, and administration of a T cell engager.

All references cited in this specification are herewith incorporated by reference with respect to their entire disclosure content and the disclosure content specifically mentioned in this specification.

### FIGURE LEGENDS

FIG. 1. Identification of modulators of B7-H3 expression by flow cytometry-based genetic screening. Dots represent individual genes; y-axis indicates Log₁₀-transformed MAGeCK robust ranking aggregation (RRA)-scores, based on the depletion or enrichment of sgRNAs in B7-H3^{high} vs B7-H3^{low} cell populations that were sorted from a genome-wide CRIPSR/Cas9 (Brunello) library of THP-1 cells.

FIG. 2. Flow cytometry analysis of B7-H3 expression in wildtype (WT) and P2RY2-deficient bulk THP-1, U937, KBM7, and K562 cells (P2RY2-3 KO and P2RY2-4 KO) in the absence or presence of ATP. Values indicate MFI relative to untreated WT cells.

FIG. 3. Flow cytometry analysis of B7-H3 expression in THP-1 cells in the absence or presence of ATP. Values indicate MFI relative to untreated cells.

FIG. 4. Effects of genetic perturbations of P2RY2 and B7-H3 on T cell activity in T cell-cancer cell co-cultures. T cells transduced with a CD33-specific CAR (CAR-T) were co-cultured with genetically edited THP-1 cells for 6-24 hours. Control cells: THP-1 cells transduced with non-targeting sgRNA and Cas9 (gNT); P2RY2 deficient cells: THP-1 cells transduced with P2RY2-targeting sgRNA and Cas9 (gP2RY2-3, gP2RY2-4); B7-H3 (CD276) deficient cells: THP-1 cells transduced with B7-H3-targeting sgRNA and Cas9 (gCD276-1); P2RY2 and B7-H3 double deficient cells: THP-1 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: THP-1 cells transduced with the open reading frame of P2RY2 (P2RY2-OE); B7-H3 overexpression cells: THP-1 cells transduced with the open reading frame of CD276 (CD276-OE). Transduced cancer cells were selected by antibiotics according to antibiotic resistance genes carried by the lentiviral constructs. Flow cytometry analysis of expression of CD25, CD69 and CD137 and production of IFNγ, IL-2 and TNFα within the CD3+CD8+ cell population is shown (values indicate the percentages of positive cells).

FIG. 5. Effects of genetic perturbations of P2RY2 and B7-H3 on T cell activity in T cell-cancer cell co-cultures. T cells transduced with a CD33-specific CAR (CAR-T) were co-cultured with genetically edited THP-1 cells for 6-24 hours. Control cells: THP-1 cells transduced with non-targeting sgRNA and Cas9 (gNT); P2RY2 deficient cells: THP-1 cells transduced with P2RY2-targeting sgRNA and Cas9 (gP2RY2-3, gP2RY2-4); B7-H3 (CD276) deficient cells: THP-1 cells transduced with B7-H3-targeting sgRNA and Cas9 (gCD276-1); P2RY2 and B7-H3 double deficient cells: THP-1 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: THP-1 cells transduced with the open reading frame of P2RY2 (P2RY2-OE); B7-H3 overexpression cells: THP-1 cells transduced with the open reading frame of CD276 (CD276-OE). Transduced cancer cells were selected by antibiotics according to antibiotic resistance genes carried by the lentiviral constructs. Flow cytometry analysis of expression of CD25 and production of IFNγ, IL-2 and TNFα within the CD3+CD8-cell population is shown (values indicate the percentages of positive cells).

FIG. 6. Effects of genetic perturbations of P2RY2 and B7-H3 on T cell activity in T cell-cancer cell co-cultures. T cells transduced with a MART-1-specific TCR (TCR-T) were co-cultured with genetically edited MART-1+BxPC3 cells for 6-24 hours. Control cells: BxPC3 cells transduced with non-targeting sgRNA and Cas9 (gNT); P2RY2 deficient cells: BxPC3 cells transduced with P2RY2-targeting sgRNA and Cas9 (gP2RY2-3, gP2RY2-4); B7-H3 (CD276) deficient cells: BxPC3 cells transduced with B7-H3-targeting sgRNA and Cas9 (gCD276-1); P2RY2 and B7-H3 double deficient cells: BxPC3 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: BxPC3 cells transduced with the open reading frame of P2RY2 (P2RY2-OE); B7-H3 overexpression cells: BxPC3 cells transduced with the open reading frame of CD276 (CD276-OE). Transduced cancer cells were selected by antibiotics according to antibiotic resistance genes carried by the lentiviral constructs. Flow cytometry analysis of expression of CD25, CD69 and CD137 and production of IFNg, IL-2 and TNFa within the CD8+ cell population is shown (values indicate the percentages of positive cells).

FIG. 7. Effects of genetic perturbations of P2RY2 and B7-H3 on cancer cell viability in T cell-cancer cell co-cultures. T cells transduced with a CD33-specific CAR (CAR-T) were co-cultured with genetically edited THP-1 cells for 6-24 hours. T cells transduced with a MART-1-specific TCR (TCR-T) were co-cultured with genetically edited or peptide-loaded cancer cells that present MART-1 epitope for 6-24 hours (The MART-1 epitope was loaded onto THP-1 cells by incubation of the cells with MART-1 peptide; BxPC3 and MCF7 cells were transduced with the MART-1 epitope). Control cells: cancer cells transduced with non-targeting sgRNA and Cas9 (gNT); P2RY2 deficient cells: cancer cells transduced with P2RY2-targeting sgRNA and Cas9 (gP2RY2-3, gP2RY2-4); B7-H3 (CD276) deficient cells: cancer cells transduced with B7-H3-targeting sgRNA and Cas9 (gCD276-1); P2RY2 and B7-H3 double deficient cells: cancer cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: cancer cells transduced with the open reading frame of P2RY2 (P2RY2-OE); B7-H3 overexpression cells: cancer cells transduced with the open reading frame of CD276 (CD276-OE). Transduced cancer cells were selected by antibiotics according to antibiotic resistance genes carried by the lentiviral constructs. For THP-1 cells, normalized counts of viable cells after co-culture with CD33-specific CAR-T cells or MART-1-specific TCR-T cells is shown. The end-point cell counts were normalized to the number of THP-1 cells in cultures without T cells. For BxPC3 cells and MCF7 cells, normalized cell viability of the cancer cells was determined by the CellTiter-Blue® Cell Viability (Promega) assay after co-culture with MART-1-specific TCR-T cells. The end-point cell viability was normalized to BxPC3 or MCF7 cells, as measured using the CellTiter-Blue® Cell Viability (Promega) assay, in cultures without T cells.

FIG. 8. Effects of P2RY2 antagonist on T cells and cancer cells in T cell-cancer cell co-cultures. T cells transduced with CD33-specific CAR (CAR-T) were co-cultured with genetically edited THP-1 cells in the absence or presence of 5 μM AR-C 118925XX (AR-C) for 6-24 hours. Control cells: THP-1 cells transduced with non-targeting sgRNA and Cas9 (gNT); P2RY2 deficient cells: THP-1 cells transduced with P2RY2-targeting sgRNA and Cas9 (gP2RY2-3, gP2RY2-4); B7-H3 (CD276) deficient cells: THP-1 cells transduced with B7-H3-targeting sgRNA and Cas9 (gCD276-1); P2RY2 and B7-H3 double deficient cells: THP-1 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: THP-1 cells transduced with the open reading frame of P2RY2 (P2RY2-OE); B7-H3 overexpression cells: THP-1 cells transduced with the open reading frame of CD276 (CD276-OE). Transduced cancer cells were selected by antibiotics according to antibiotic resistance genes carried by the lentiviral constructs. Flow cytometry analysis of expression of production of IFNγ and TNFα within the CD3+CD8+ cell population is shown (values indicate the percentages of positive cells). For the viability of THP-1 cells, normalized cell counts of viable cells after co-culture with CD33-specific CAR-T cells is shown. The end-point cell counts were normalized to the number of THP-1 cells in cultures without T cells.

FIG. 9. Effect of P2RY2 antagonist and agonist on cancer cells in T cell-cancer cell cocultures. T cells transduced with CD33-specific CAR (CAR-T) were co-cultured with genetically edited THP-1 cells in the absence or presence of 5 uM AR-C 118925XX (AR-C) or 100 uM Diquafosol for 24 hours. Control cells THP-1 cells transduced with non-targeting sgRNA and Cas9 (Ctrl.); P2RY2 overexpression cells: THP-1 cells transduced with the open reading frame of P2RY2 (P2RY2-OE). Transduced cancer cells were selected by antibiotics according to antibiotic resistance genes carried by the lentiviral constructs. Normalized cell counts of viable cells after co-culture with CD33-specific CAR-T cells is shown. The end-point cell counts were normalized to the number of THP-1 cells in cultures without T cells.

FIG. 10. Effects of P2RY2 inhibition and PD-L1 immune checkpoint inhibition on T cells in T cell-cancer cell co-cultures. T cells transduced with a MART-1-specific TCR (TCR-T) were co-cultured with MART-1⁺BxPC3 cells in the presence of 5 μM AR-C 118925XX (AR-C), 10 μg/ml atezolizumab, 10 μg/ml durvalumab, or their combinations for 6-24 hours. Untreated cells served as the control. Flow cytometry analysis of expression of CD25, CD69 and CD137 within the CD8+ MART-1-specific TCR+ population is shown (values indicate the percentages of positive cells). Data represent mean±s.d. of triplicates, and were analyzed by one-way ANOVA with multiple comparisons *p≤0.0013.

FIG. 11. Effects of P2RY2 inhibition and PD-L1 immune checkpoint inhibition on cancer cells in T cell-cancer cell co-cultures. T cells transduced with a MART-1-specific TCR (TCR-T) were co-cultured with MART-1+BxPC3 cells in the presence of 5 μM AR-C 118925XX (AR-C), 10 μg/ml atezolizumab, 10 μg/ml durvalumab, or their combinations for 6-24 hours. Untreated cells served as the control. Normalized cell viability of the BxPC3 cells was determined by the CellTiter-Blue® Cell Viability (Promega) assay after co-culture with MART-1-specific TCR-T cells. The end-point cell viability was normalized to BxPC3 cells in cultures without T cells. Data represent mean±s.d. of triplicates, were analyzed by one-way ANOVA with multiple comparisons *p≤0.0013.

FIG. 12. Effects of genetic perturbations of P2RY2 and B7-H3 on T cell activity in T cell-cancer cell co-cultures. T cells transduced with a CEA-specific CAR (CAR-T) were co-cultured with genetically edited SW480 cells for 24 hours. Control cells: SW480 cells transduced with non-targeting sgRNA and Cas9 (gNT); P2RY2 deficient cells: SW480 cells transduced with P2RY2-targeting sgRNA and Cas9 (gP2RY2-3, gP2RY2-4); B7-H3 (CD276) deficient cells: SW480 cells transduced with B7-H3-targeting sgRNA and Cas9 (gCD276-1); P2RY2 and B7-H3 double deficient cells: SW480 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: SW480 cells transduced with the open reading frame of P2RY2 (P2RY2-OE); B7-H3 overexpression cells: SW480 cells transduced with the open reading frame of CD276 (CD276-OE). Transduced cancer cells were selected by antibiotics according to antibiotic resistance genes carried by the lentiviral constructs. Flow cytometry analysis of expression of CD25, CD69 and CD137 and production of IFNγ, IL-2 and TNFα within the CD3+CD8+ cell population is shown (values indicate the percentages of positive cells).

FIG. 13. Effects of genetic perturbations of P2RY2 and B7-H3 on T cell activity in T cell-cancer cell co-cultures. T cells transduced with a CEA-specific CAR (CAR-T) were co-cultured with genetically edited SW480 cells for 24 hours. Control cells: SW480 cells transduced with non-targeting sgRNA and Cas9 (gNT); P2RY2 deficient cells: SW480 cells transduced with P2RY2-targeting sgRNA and Cas9 (gP2RY2-3, gP2RY2-4); B7-H3 (CD276) deficient cells: SW480 cells transduced with B7-H3-targeting sgRNA and Cas9 (gCD276-1); P2RY2 and B7-H3 double deficient cells: SW480 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: SW480 cells transduced with the open reading frame of P2RY2 (P2RY2-OE); B7-H3 overexpression cells: SW480 cells transduced with the open reading frame of CD276 (CD276-OE). Transduced cancer cells were selected by antibiotics according to antibiotic resistance genes carried by the lentiviral constructs. Flow cytometry analysis of expression of CD25, CD69 and CD137 and production of IFNγ, IL-2 and TNFα within the CD3+CD4+ cell population is shown (values indicate the percentages of positive cells).

FIG. 14. Effects of genetic perturbations of P2RY2 and B7-H3 on T cell activity in T cell-cancer cell co-cultures. T cells transduced with a MART-1-specific TCR (TCR-T) were co-cultured with genetically edited MART-1+MIA PaCa-2 cells for 16 hours. Control cells: MIA PaCa-2 cells transduced with non-targeting sgRNA and Cas9 (gNT); P2RY2 deficient cells: MIA PaCa-2 cells transduced with P2RY2-targeting sgRNA and Cas9 (gP2RY2-3, gP2RY2-4); B7-H3 (CD276) deficient cells: MIA PaCa-2 cells transduced with B7-H3-targeting sgRNA and Cas9 (gCD276-1); P2RY2 and B7-H3 double deficient cells: MIA PaCa-2 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: MIA PaCa-2 cells transduced with the open reading frame of P2RY2 (P2RY2-OE); B7-H3 overexpression cells: MIA PaCa-2 cells transduced with the open reading frame of CD276 (CD276-OE). Transduced cancer cells were selected by antibiotics according to antibiotic resistance genes carried by the lentiviral constructs. Flow cytometry analysis of expression of CD25, CD69 and CD137 and production of IFNγ and IL-2 within the CD3+CD8+ cell population is shown (values indicate the percentages of positive cells).

FIG. 15. Effects of genetic perturbations of P2RY2 and B7-H3 on T cell activity in T cell-cancer cell co-cultures. T cells transduced with a MART-1-specific TCR (TCR-T) were co-cultured with genetically edited MART-1+MIA PaCa-2 cells for 16 hours. Control cells: MIA PaCa-2 cells transduced with non-targeting sgRNA and Cas9 (gNT); P2RY2 deficient cells: MIA PaCa-2 cells transduced with P2RY2-targeting sgRNA and Cas9 (gP2RY2-3, gP2RY2-4); B7-H3 (CD276) deficient cells: MIA PaCa-2 cells transduced with B7-H3-targeting sgRNA and Cas9 (gCD276-1); P2RY2 and B7-H3 double deficient cells: MIA PaCa-2 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: MIA PaCa-2 cells transduced with the open reading frame of P2RY2 (P2RY2-OE); B7-H3 overexpression cells: MIA PaCa-2 cells transduced with the open reading frame of CD276 (CD276-OE). Transduced cancer cells were selected by antibiotics according to antibiotic resistance genes carried by the lentiviral constructs. Flow cytometry analysis of expression of CD25 and CD69 and production of IFNγ and IL-2 within the CD3+CD4+ cell population is shown (values indicate the percentages of positive cells).

FIG. 16. Effects of genetic perturbations of P2RY2 and B7-H3 on T cell activity in T cell-cancer cell co-cultures. T cells transduced with a MART-1-specific TCR (TCR-T) were co-cultured with genetically edited MART-1+ MDA-MB-231 cells for 21 hours. Control cells: MDA-MB-231 cells transduced with non-targeting sgRNA and Cas9 (gNT); P2RY2 deficient cells: MDA-MB-231 cells transduced with P2RY2-targeting sgRNA and Cas9 (gP2RY2-3, gP2RY2-4); B7-H3 (CD276) deficient cells: MDA-MB-231 cells transduced with B7-H3-targeting sgRNA and Cas9 (gCD276-1); P2RY2 and B7-H3 double deficient cells: MDA-MB-231 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: MDA-MB-231 cells transduced with the open reading frame of P2RY2 (P2RY2-OE); B7-H3 overexpression cells: MDA-MB-231 cells transduced with the open reading frame of CD276 (CD276-OE). Transduced cancer cells were selected by antibiotics according to antibiotic resistance genes carried by the lentiviral constructs. Flow cytometry analysis of expression of CD25 and CD69 within the CD3+CD8+ or CD3+CD4+ cell population is shown (values indicate the percentages of positive cells).

FIG. 17. Effects of genetic perturbations of P2RY2 and B7-H3 on cancer cell viability in CAR-T cell-cancer cell co-cultures. T cells transduced with a CEA-specific CAR (CAR-T) were co-cultured with genetically edited SW480 cells for 48 h. Control cells: cancer cells transduced with non-targeting sgRNA and Cas9 (gNT); P2RY2 deficient cells: cancer cells transduced with P2RY2-targeting sgRNA and Cas9 (gP2RY2-3, gP2RY2-4); B7-H3 (CD276) deficient cells: cancer cells transduced with B7-H3-targeting sgRNA and Cas9 (gCD276-1); P2RY2 and B7-H3 double deficient cells: cancer cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: cancer cells transduced with the open reading frame of P2RY2 (P2RY2-OE); B7-H3 overexpression cells: cancer cells transduced with the open reading frame of CD276 (CD276-OE). Transduced cancer cells were selected by antibiotics according to antibiotic resistance genes carried by the lentiviral constructs. Normalized cell viability of the cancer cells was determined by the CellTiter-Blue® Cell Viability (Promega) assay after co-culture with MART-1-specific TCR-T cells. The end-point cell viability was normalized to SW480 cells, as measured using the CellTiter-Blue® Cell Viability (Promega) assay, in cultures without T cells.

FIG. 18. Effects of genetic perturbations of P2RY2 and B7-H3 on cancer cell viability in TCR-T cell-cancer cell co-cultures. T cells transduced with a MART-1-specific TCR (TCR-T) were co-cultured with genetically edited MART-1+ MDA-MB-231/MIA PaCa-2 cells for 48 hours. MIA PaCa-2 and MDA-MB-231 cells were transduced with the MART-1 epitope.

Control cells: cancer cells transduced with non-targeting sgRNA and Cas9 (gNT); P2RY2 deficient cells: cancer cells transduced with P2RY2-targeting sgRNA and Cas9 (gP2RY2-3, gP2RY2-4); B7-H3 (CD276) deficient cells: cancer cells transduced with B7-H3-targeting sgRNA and Cas9 (gCD276-1); P2RY2 and B7-H3 double deficient cells: cancer cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: cancer cells transduced with the open reading frame of P2RY2 (P2RY2-OE); B7-H3 overexpression cells: cancer cells transduced with the open reading frame of CD276 (CD276-OE). Transduced cancer cells were selected by antibiotics according to antibiotic resistance genes carried by the lentiviral constructs. Normalized cell viability of the cancer cells was determined by the CellTiter-Blue® Cell Viability (Promega) assay after co-culture with MART-1-specific TCR-T cells. The end-point cell viability was normalized to MIA PaCa-2 and MDA-MB-231 cells, as measured using the CellTiter-Blue® Cell Viability (Promega) assay, in cultures without T cells.

FIG. 19. Effects of P2RY2 agonist and P2RY2 antagonist on T cell activity in T cell-cancer cell co-cultures. T cells transduced with a CEA-specific CAR (CAR-T) were co-cultured with genetically edited SW480 cells for 24 hours in the absence or presence of 40 μM ATP, 200 μM ATPγS, 10 μM Diquafosol, 5 μM Denufosol and 10 μM AR-C 118925XX (AR-C). Control cells: SW480 cells transduced with non-targeting sgRNA and Cas9 (gNT); P2RY2 deficient cells: SW480 cells transduced with P2RY2-targeting sgRNA and Cas9 (gP2RY2-3, gP2RY2-4); B7-H3 (CD276) deficient cells: SW480 cells transduced with B7-H3-targeting sgRNA and Cas9 (gCD276-1); P2RY2 and B7-H3 double deficient cells: SW480 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: SW480 cells transduced with the open reading frame of P2RY2 (P2RY2-OE); B7-H3 overexpression cells: SW480 cells transduced with the open reading frame of CD276 (CD276-OE). Transduced cancer cells were selected by antibiotics according to antibiotic resistance genes carried by the lentiviral constructs. Flow cytometry analysis of expression of CD25 and CD69 and production of IFNγ within the CD3+CD8+ or CD3+CD4+ cell population is shown (values indicate the percentages of positive cells).

FIG. 20. Effects of P2RY2 agonist and P2RY2 antagonist on cancer cell viability in T cell-cancer cell co-cultures. T cells transduced with a CEA-specific CAR (CAR-T) were co-cultured with genetically edited SW480 cells for 48 hours in the absence or presence of 40 μM ATP, 20 μM ATPγS, 10 μM Diquafosol, 5 μM Denufosol and 10 μM AR-C 118925XX (AR-C). Control cells: SW480 cells transduced with non-targeting sgRNA and Cas9 (gNT); P2RY2 deficient cells: SW480 cells transduced with P2RY2-targeting sgRNA and Cas9 (gP2RY2-3, gP2RY2-4); B7-H3 (CD276) deficient cells: SW480 cells transduced with B7-H3-targeting sgRNA and Cas9 (gCD276-1); P2RY2 and B7-H3 double deficient cells: SW480 cells transduced with P2RY2-targeting sgRNA, B7-H3-targeting sgRNA, and Cas9 (gCD276-1 gP2RY2-3 DKO, gCD276-1 gP2RY2-4 DKO); P2RY2 overexpression cells: SW480 cells transduced with the open reading frame of P2RY2 (P2RY2-OE); B7-H3 overexpression cells: SW480 cells transduced with the open reading frame of CD276 (CD276-OE). Transduced cancer cells were selected by antibiotics according to antibiotic resistance genes carried by the lentiviral constructs. Normalized cell viability of the SW480 cells was determined by the CellTiter-Blue® Cell Viability (Promega) assay after co-culture with CEA CAR-T cells. The end-point cell viability was normalized to SW480 cells, as measured using the CellTiter-Blue® Cell Viability (Promega) assay, in cultures without T cells.

The following Examples shall merely illustrate the invention. They shall not be construed, whatsoever, to limit the scope of the invention.

### EXAMPLE 1: FLOW CYTOMETRY-BASED CRISPR-CAS9 SCREEN FOR MODULATORS OF B7-H3

To identify novel modulators of B7-H3, we used a forward genetic screening approach in human THP-1 cells. THP-1 cells spontaneously express B7-H3 at the cell surface at levels that can be detected with antibodies.

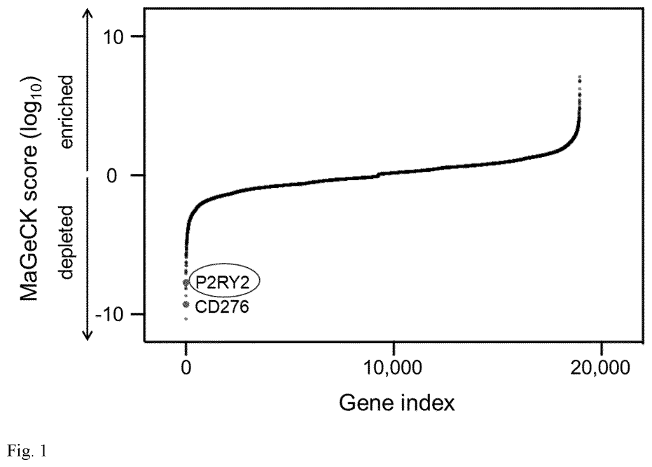

We introduced a genome-wide CRISPR knockout library (Doench et al., Nat Biotechnol 34, 184-191 (2016)) into THP-1 cells by lentiviral transduction to create a pool of mutant cells, selected the transduced cells, expanded the cells, and incubated them using antibodies specific for the B7-H3 protein. The pool of antibody-incubated cells renders a near-normal distribution of signal intensity when analyzed by flow cytometry. Using fluorescence-activated cell sorting (FACS), Cas9/sgRNA-modified cells that display particularly strong or particularly weak B7-H3 signal were separately isolated, and sgRNA abundance in both cell pools, was subsequently compared (FIG. 1).

### Material and Method

To generate a whole genome mutant library of THP-1 cells, a Cas9 expressing THP-1 clone (c135) was lentivirally transduced with the Human Brunello CRISPR Genome-Wide Knockout Library in the lentiGuide-Puro vector (Doench et al., Nat Biotechnol 34, 184-191 (2016)) at a coverage of 1,500-2,000 folds, with an infection rate of approximately 40%, and cells were selected with 1.0 μg ml-1 of puromycin for at least 10 days. To screen for modulators of B7-H3 expression on THP-1 cells, 3×10⁸ library cells were collected and washed once with PBS and stained with Near-IR Dead Cell Stain (ThermoFisher) in PBS for 10 min at 4° C. while protected from light. After 2 washes in PBS containing 0.5% (w/v) BSA (Sigma) and 0.2% (w/v) sodium azide (Sigma) (FACS buffer), cells were stained with unconjugated anti-B7-H3 MIH42 (Biolegend) at a dilution of 0.1 g ml⁻¹ for 30 min in FACS buffer at a concentration of 1×10⁷ cells/mL, at 4° C. while protected from light. After 2 washes with FACS buffer, cells were stained with AF488-conjugated goat anti-mouse IgG antibody (A28175, ThermoFisher) at a dilution of 2 g ml⁻¹ for 30 min in FACS buffer at 4° C. while protected from light. After 2 washes with FACS buffer, cells were fixed with IC Fixation Buffer (eBioscience), according to the manufacturer's protocol. Next, cells were passed through a 40 m strainer (BD Falcon) and subjected to a FACS sorter to separately collect the cell populations that display the highest and lowest B7-H3 signal, both constituting approximately 5-10% of the total cell pool.

The two sorted cell populations were pelleted by centrifugation (2,000 rpm, 10 minutes), subjected to genomic DNA isolation using Qiagen DNA mini kit (Qiagen). As the cells were fixed, to facilitate the de-crosslink, cell pellets were resuspended in PBS and, after the addition of Proteinase K and buffer AL (Qiagen), incubated at 56° C. on a Thermmixer (Eppendorf) for at least 12 hours. DNA was isolated according to the manufacturer's instructions and the concentration of the DNA was determined by Nanodrop2000 spectrophotometer (Thermo Fisher). gRNA integrated in the genomic DNA of the sorted cell populations were separately amplified by a two-step PCR using NEBnext High Fidelity Master Mix (New England Biolab) with the following primer sets.

Step#1 PCR: forward primer
(ACACTCTTTCCCTACACGACGCTCTTCCGATCTGTAGCCGGCTTTATAT
ATCTTGTGGAAAGGACG (SEQ ID NO: 1)
or
ACACTCTTTCCCTACACGACGCTCTTCCGATCTTACAAGGGCTTTATATA
TCTTGTGGAAAGGACG (SEQ ID NO: 2))
and
reverse primer
(GTGACTGGAGTTCAGACGTGTGCTCTTCCGATCTACTGACGGGCACCGG
AGCCAATTCC (SEQ ID NO: 3))
Step #2 PCR: forward primer
(AATGATACGGCGACCACCGAGATCTACACTCTTTCCCTACACGACGCTC
TTCCGATCT SEQ ID NO: 4)
and
reverse primer
(CAAGCAGAAGACGGCATACGAGATTGGTCAGTGACTGGAGTTCAGACGT
GTGCTCTTCCGATCT (SEQ ID NO: 5)).

The cycling condition of PCR #1: 30 seconds at 98° C., 18×(10 seconds at 98° C., 30 seconds at 60° C., 30 seconds at 72° C.), 2 min at 72° C., 4° C. end. The PCR #1 products from each sorted cell population were pooled and 4×2 μl of the pooled PCR products were used as templates in 4×50 μl PCR #2 reactions. The cycling condition of PCR #2: 30 seconds at 98° C., 12×(10 seconds at 98° C., 30 seconds at 60° C., 30 seconds at 72° C.), 2 min at 72° C., 4° C. end. PCR #2 products were purified using QIAquick PCR purification kit (Qiagen) according to the manufacturer's instructions. The resulting PCR amplicons were analyzed by Bioanalyzer (Agilent) and samples were equimolarly pooled, sequenced by HiSeq 2500 (Illumina, High Output Mode, Single-Read, 65 bp), and analyzed by MAGeCK (Li et al., Genome Biology. 2014).

### Results

The results of the genetic screen for modulators of surface B7-H3 expression in THP-1 cells are shown in FIG. 1. As expected, sgRNAs targeting the CD276 gene, encoding B7-H3 itself, were highly enriched in B7-H3low cells, underpinning the reliability of the genetic screen. In addition, this screen identified P2RY2, encoding the purinergic receptor P2RY2, as a positive regulator of B7-H3.

### EXAMPLE 2: P2RY2 AND B7-H3 EXPRESSION

We set out to validate the involvement of P2RY2 in modulating surface B7-H3 levels by transducing various cell lines with lentiviral vectors encoding Cas9 and sgRNA targeting the P2RY2 gene.

### Material and Method

THP-1 cells, U937 cells, K562 cells, and KBM7 cells were transduced with a lentiviral vector encoding Cas9 along with a Blasticidin S resistance cassette (Blasticidin D deaminase). One day after the transduction, cells were selected with 50 microgram/milliliter Blasticidin S Hydrochloride for 72-48 hours. Next, surviving cells were transduced with one of the lentiviral vectors encoding sgRNA targeting the sequences 5′-CTGGTCTATTACTACGCCCG-3′ (SEQ ID NO:6) and 5′-TGGGCTGTGTCTGAACGCCG-3′ (SEQ ID NO:7) on the coding strand of P2RY2 gene, along with a puromycin resistance cassette (puromycin N-acetyltransferase). One day after the transduction, cells were selected with 2 microgram/milliliter Puromycin dihydrochloride for 48-96 hours. Selected cells were expanded, subjected to genomic DNA isolation, and analyzed for deleterious editing of the P2RY2 locus by PCR (using primers 5′-GATGAGCTGGGCTACAGGTG-3′ (SEQ ID NO:8) and 5′-GTCGTAAGACGCCCAGACAC-3′ (SEQ ID NO:9)) and Sanger sequencing of the PCR products using primer 5′-GATGAGCTGGGCTACAGGTG-3′ (SEQ ID NO:8) for the sgRNA(5′-CTGGTCTATTACTACGCCCG-3′, SEQ ID NO:6)) transduced cell and 5′-GTCGTAAGACGCCCAGACAC-3′ (SEQ ID NO:9) for the sgRNA(5′-TGGGCTGTGTCTGAACGCCG-3′, SEQ ID NO:7) transduced cell.

The polyclonal cells transduced with Cas9 and sgRNA targeting P2RY2, and wildtype cells were cultured in the absence or presence of ATP or UTP (ligands of P2RY2, Sigma) for 24 hours, stained with anti-B7-H3 antibody and analyzed by flow cytometry.

### Results

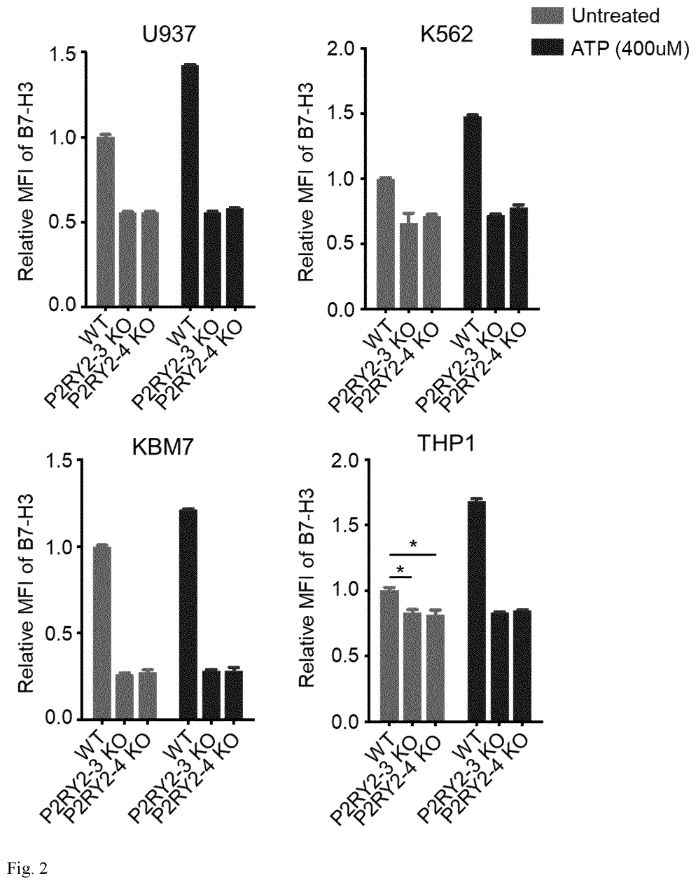

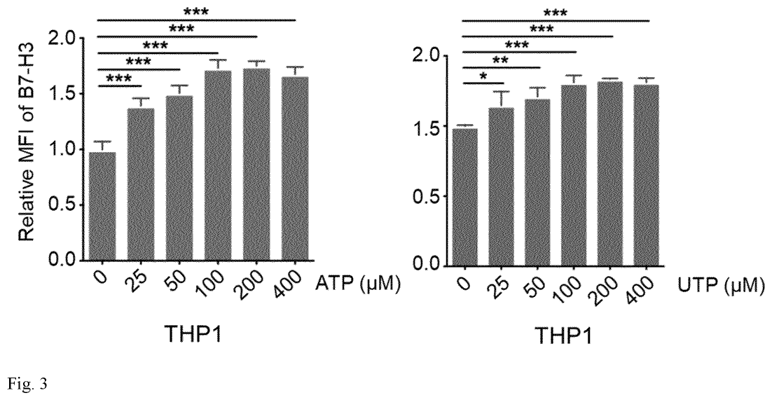

CRISPR/Cas9-mediated knock-out of P2RY2 resulted in a decrease in B7-H3 surface levels in THP-1 cells, U937 cells, K562 cells, and KBM7 cells (FIG. 2). P2RY2 has previously been identified as a receptor of extracellular ATP and UTP (Burnstock et al., Purigergic Signal 9, 491-540 (2013)). We next stimulated P2RY2 proficient cells with ATP or UTP and observed increased B7-H3 expression in a dose-dependent manner (FIG. 3). Notably, ATP is present at very high concentrations in the interstitium of tumors, but at much lower concentrations in healthy tissues (Di Virgilio et al., Oncogene 36, 293-303 (2017); Kepp et al., Immunol Rev. 280(1): 83-92 (2017); Pellegatti et al., PLOS one 3(7): e2599 (2008)). We next tested whether P2RY2 regulates ATP-induced B7-H3 surface expression and observed that the ATP-induced-B7-H3 expression was abrogated upon P2RY2 disruption (FIG. 2).

### EXAMPLE 3: P2RY2 AND T CELL ACTIVITY

### Material and Method

MART-1 specific T cells were generated as described (Mezzadra et al., Nature 549, 106-110 (2017); Jorritsma et al., Blood 110 (10): 3564-3572. (2007)). To generate CD33-specific chimeric antigen receptor (CAR) T cells, a CD33Hul95-CD28Z CAR (described in WO 2019/178382) cassette was cloned into a retroviral vector, and the resulting virus was used to transduce T cells as described (Mezzadra et al., Nature 549, 106-110 (2017); Jorritsma et al., Blood 110 (10): 3564-3572. (2007)).

For cancer cells that endogenously express HLA-A2, including THP-1 and MCF7, the MART-1 epitope was loaded on cancer cells by incubation of modified MART-1 peptides (ELAGIGILTV, SEQ ID NO:12) with the cells or by lentiviral transduction. For the latter, a gene fragment encoding the MART-1 epitope (ELAGIGILTV, SEQ ID NO:12) followed by P2A-RFP was cloned into a lentiviral vector, and the resulting virus was used to transduce cancer cells. For cancer cells that do not express endogenous HLA-A2 including BxPC3 cells, a gene fragment encoding the MART-1 epitope (ELAGIGILTV, SEQ ID NO:12) followed by P2A-HLA-A2/B2M was cloned into a lentiviral vector, which was used to generate MART-1V cancer cells by transduction.

Cas9 and sgRNA targeting P2RY2 (5′-CTGGTCTATTACTACGCCCG-3′ (SEQ ID NO:6) and 5′-TGGGCTGTGTCTGAACGCCG-3′ (SEQ ID NO:7)), CD276 (5′-CACAGGGCAACGCATCCCTG-3′, SEQ ID NO:10), or both were introduced into the cancer cells by lentiviral transduction as described in Example 2. sgRNA(5′-GTATTACTGATATTGGTGGG-3′, SEQ ID NO:11) that does not recognize any sequence in the human genome served as a control. The open reading frames of transcripts of P2RY2(ENST00000393597.7) or CD276(ENST00000318443.10), were ordered as a codon optimized gBlock Gene Fragment (Integrated DNA Technologies or Twist Bioscience) and cloned into a lentiviral vector containing a blasticidin selection cassette. Constructs were verified by Sanger sequencing.

MART1-specific CD8+ T cells and MART1 epitope-expressing cancer cells were mixed at a ratio of 1:16 to 1:4. Next, cells were centrifugated at 1,600 g for 3 min and incubated at 37° C. and 5% CO₂ for 12-72 hours. T cell activation was assessed by surface staining with anti-CD137-BV421 (4-1BB, Biolegend), anti-CD69-APC/Fire™ 750 (FN50, Biolegend), anti-CD25-AF700 (BC96, Biolegend) antibodies and intracellular staining with anti-IL-2-PE/Cy7 (MQ1-17H12, Biolegend), anti-IFN-7-BV421 (4S.B3, Biolegend), anti-TNF-α-BV785 (MAb11, Biolegend) antibodies, followed by flow cytometry analysis. For intracellular staining, brefeldin A (Biolegend) was added to the cell culture medium 4 h before cells were harvested. The viability of cancer cells that grow in suspension including THP-1 cells was assessed by flow cytometry-based absolute cell counting using AccuCount beads (Spherotech). For adherent cancer cells including BxPC3 and MCF7, the cell viability was quantified using CellTiter-Blue Cell Viability Assay (Promega).

### Result

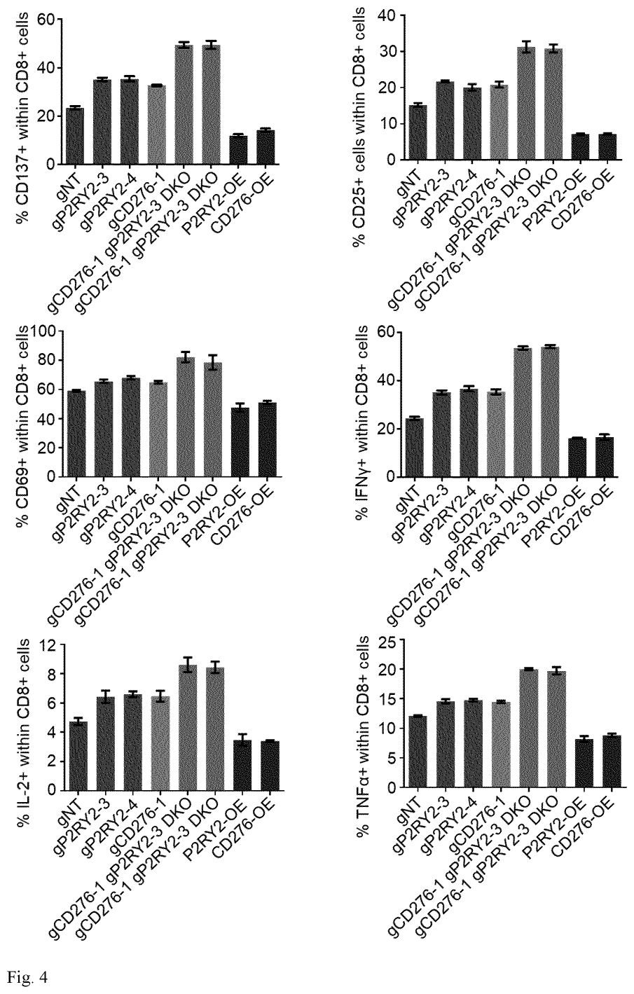

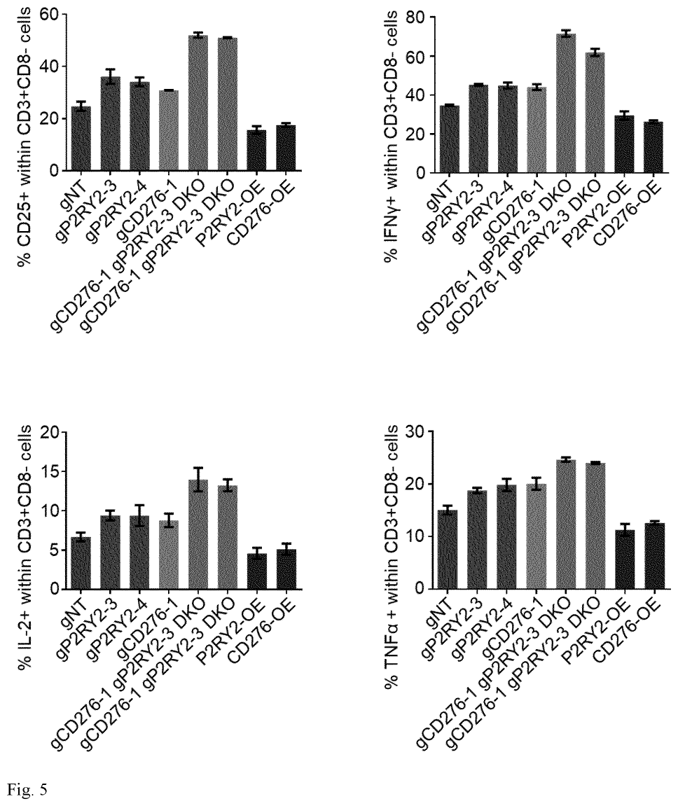

To determine the effects of P2RY2 on T cells and cancer cells in in vitro models of T cell-based immunotherapies, we engineered human T cells that are derived from peripheral blood with the CD33-specific CAR or a MART-1 specific TCR, and assessed T cell activation in the presence of antigen-expressing cancer cells that carry Cas9/sgRNA targeting P2RY, B7-H3, or both, or antigen-expressing cancer cells that overexpress P2RY2 or B7-H3. When CD33-specific CAR transduced T cells were co-cultured with THP-1 cells that endogenously express CD33, P2RY2 and B7-H3 deletion in THP-1 cells resulted in elevated T cell activation, as evidenced by increased expression of T cell activation markers CD25, CD69 and CD137, and also increased production of the cytokines IFNγ, IL-2 and TNFα in CD3+CD8+ T cells. In contrast, overexpression of either P2RY2 or B7-H3 in tumor cells resulted in impaired CD3+CD8+ T cell activation and cytokine production (FIG. 4). Moreover, in CD3+CD8− T cell population, individual deletion of P2RY2− or B7-H3 in THP-1 cells led to increased expression of T cell activation marker including CD25, IFNγ, IL-2 and TNFα while overexpression of P2RY2 or B7-H3 reduces T cell activation (FIG. 5).

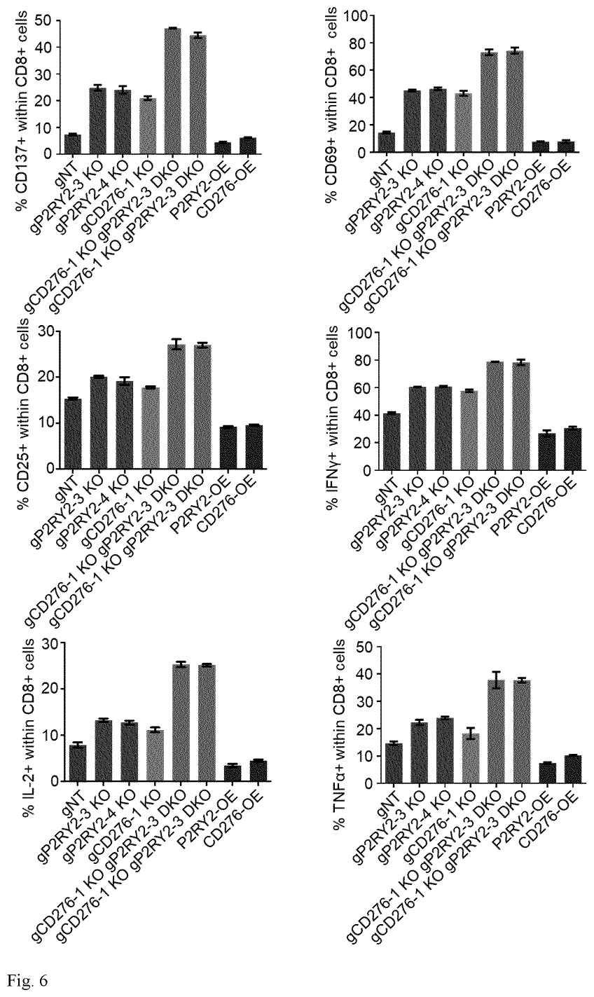

Notably, simultaneous deletion of P2RY2 and B7-H3 in THP-1 increased the expression of these T cell activation markers (CD25, IFNγ, IL-2 and TNFα) to higher levels than individual deletion of P2RY2 or B7-H3 in both CD3+CD8+ and CD3+CD8− T cells (FIG. 4, 5). Similar effects were observed in the co-culture of MART-1-specific TCR transduced T cells with MART-1 epitope transduced BxPC3 cells (FIG. 6).

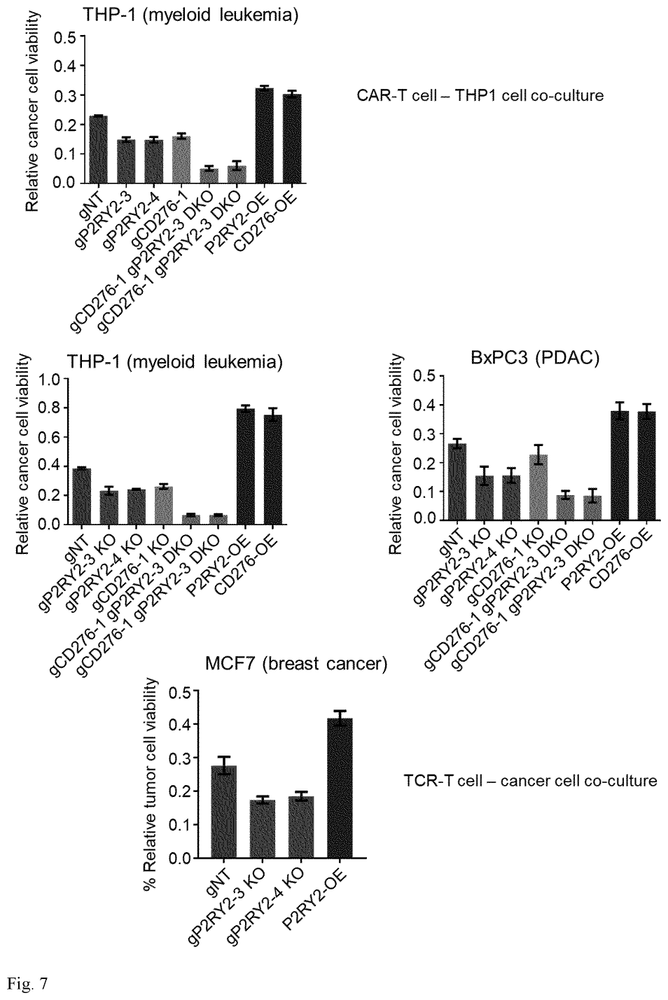

In line with these data, P2RY2 or B7-H3 disruption in cancer cells resulted in increased tumor cell killing by tumor-reactive CAR-T or TCR-T cells in myeloid leukemia cell (THP-1), pancreatic cancer cell (BxPC3), and breast cancer cell (MCF7) models. Moreover, combinatorial deletion of P2RY2 and B7-H3 significantly improved the tumoricidal effect of T cells. In contrast, overexpression of P2RY2 or B7-H3 in cancer cells conferred resistance to the tumor-reactive T cells (FIG. 7). These results suggest that P2RY2 suppresses T cell activation through both B7-H3-dependent and B7-H3-independent mechanisms and combinatorial deletion of P2RY2 and B7-H3 further improves T cell-mediated anticancer effects over deletion of either molecule alone.

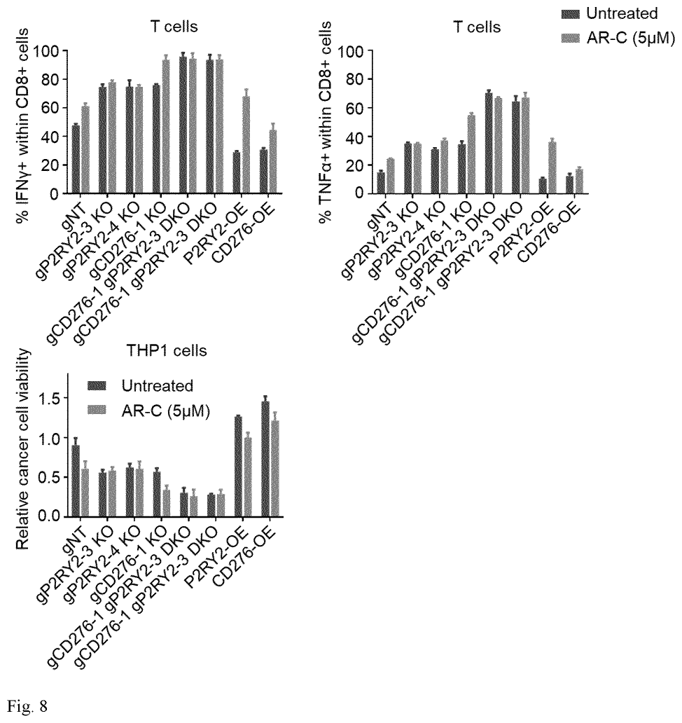

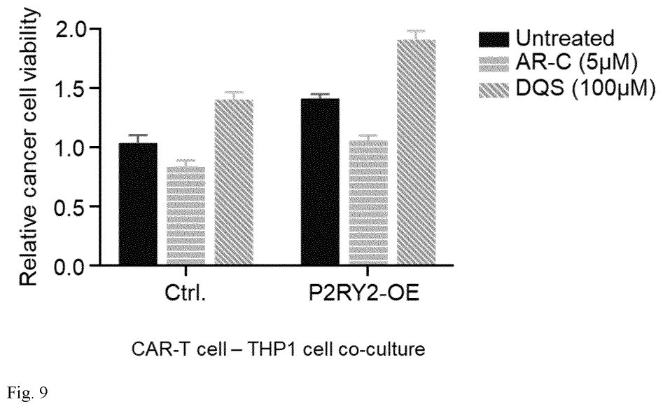

Furthermore, we found that pharmacological inhibition of P2RY2 using an inhibitory tool compound (AR-C 118925XX) increased the production of IFNγ and TNFα by T cells and reduced the viability of P2RY2 proficient THP-1 cells when CD33-specific CAR transduced T cells were co-cultured with THP-1 cells (FIG. 8). Additionally, upon P2RY2 deletion, AR-C 118925XX does not influence the production of IFNγ and TNFα in T cell or the viability of tumor cells in CAR-T cell-THP-1 cell coculture assays, demonstrating that the compound functions through P2RY2. In contrast, a P2RY2 agonist (Diquafosol) confers resistance to T cell-mediated cytotoxicity in THP-1 cells (FIG. 9).

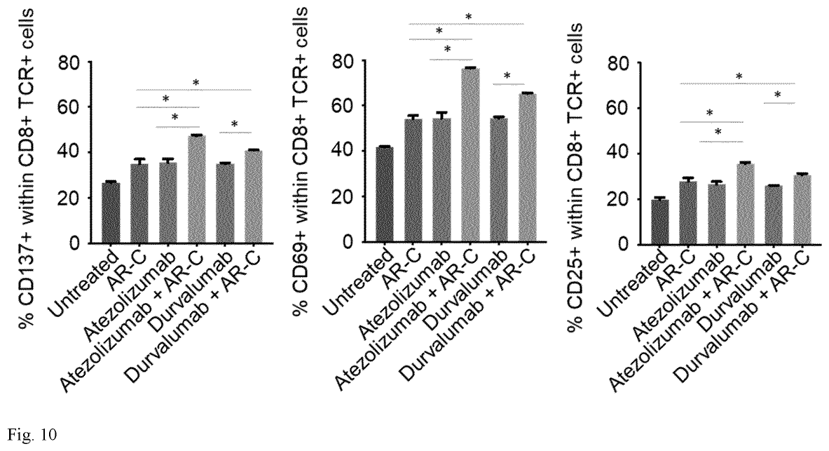

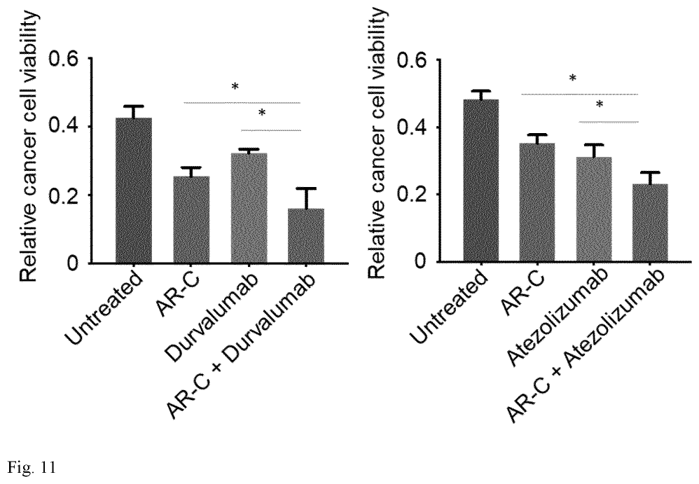

Besides the co-inhibition of P2RY2 and the B7-H3 immune checkpoint, combinations of a P2RY2 inhibitor and a blocking antibody of the PD-L1-PD-1 immune checkpoint were tested. When MART-1-specific TCR transduced T cells were cultured in the presence of MART-1-epitope transduced BxPC3 cells, inhibition of P2RY2 by AR-C 118925XX or blockade of the PD-L1-PD-1 immune checkpoint by atezolizumab or durvalumab elevated the expression of CD137, CD69, and CD25 on T cells, while combinatorial P2RY2 and a PD-L1 inhibition further increased the expression of T cell activation markers (FIG. 10). In line with this, P2RY2 inhibition by AR-C 118925XX or PD-L1 blockade by atezolizumab or durvalumab enhanced the tumoricidal effect of T cells as evidenced by reduced viability of BxPC3 cells, while combined P2RY2 and PD-L1 inhibition resulted in an improved antitumor effect, as compared to both individual treatments (FIG. 11).

Together, these data demonstrate that P2RY2 expression on cancer cells inhibits both T cell activation and T cell-mediated cytotoxicity, and that the modulation of P2RY2 activity can be exploited to modulate T cell activity in immunotherapies.

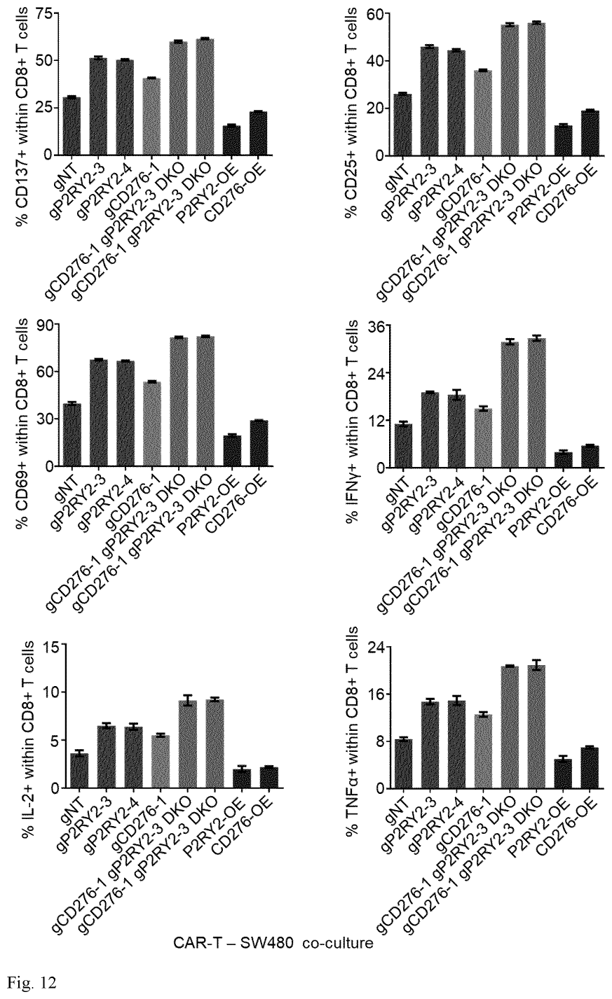

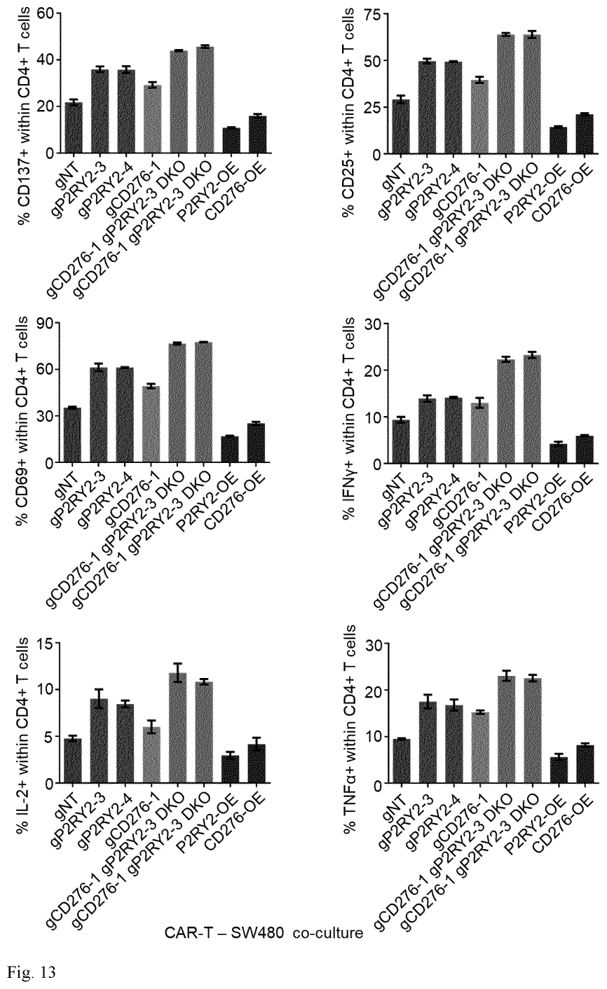

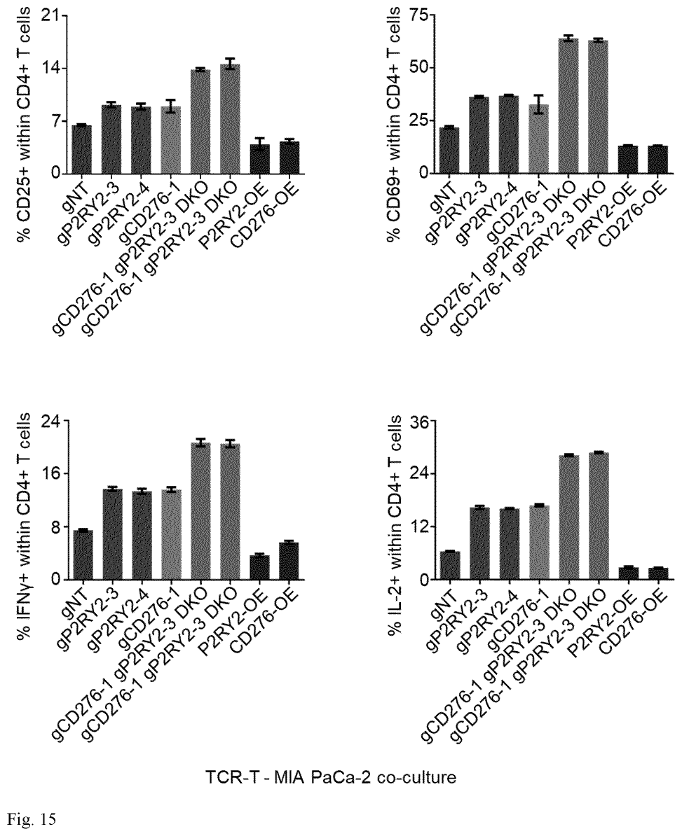

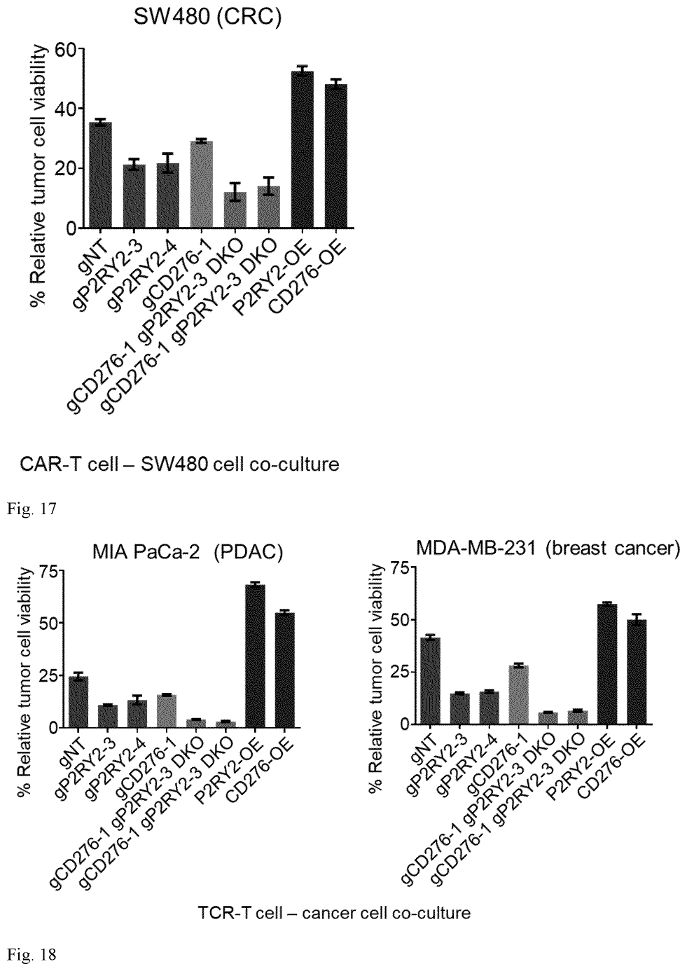

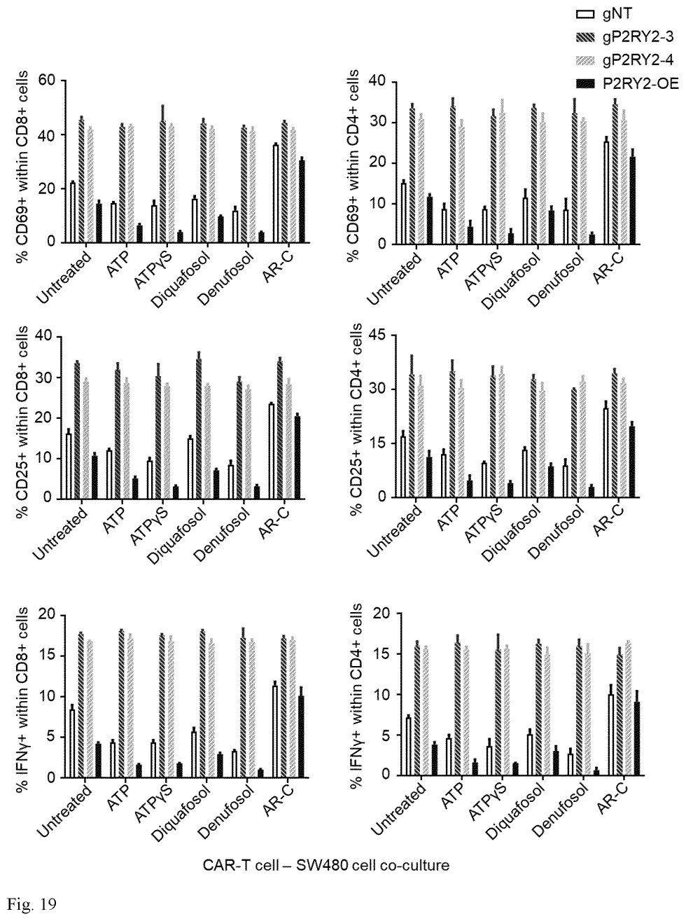

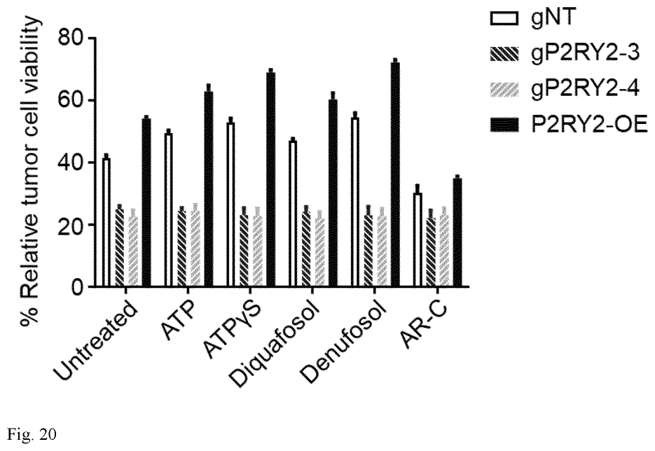

### EXAMPLE 4: P2RY2 AND T CELL ACTIVITY (FIGS. 12 TO 20)

### Material and Method

MART-1 specific T cells were generated as described (Mezzadra et al., Nature 549, 106-110 (2017); Jorritsma et al., Blood 110 (10): 3564-3572. (2007)). To generate CD33-specific chimeric antigen receptor (CAR) T cells and CEA-specific CAR-T cells, a CD33Hul95-CD28Z CAR (described in WO 2019/178382) cassette and MFE-23 (described in US20050147614A1)-CD28Z CAR were separately cloned into retroviral vectors, and the resulting virus was used to transduce T cells as described (Mezzadra et al., Nature 549, 106-110 (2017); Jorritsma et al., Blood 110 (10): 3564-3572. (2007)).

For cancer cells that endogenously express HLA-A2, including THP-1, MDA-MB-231 and MCF7, the MART-1 epitope was loaded on cancer cells by incubation of modified MART-1 peptides (ELAGIGILTV, SEQ ID NO:12) with the cells or by lentiviral transduction. For the latter, a gene fragment encoding the MART-1 epitope (ELAGIGILTV, SEQ ID NO:12) followed by P2A-RFP was cloned into a lentiviral vector, and the resulting virus was used to transduce cancer cells. For cancer cells that do not express endogenous HLA-A2 including BxPC3 and MIA PaCa-2 cells, a gene fragment encoding the MART-1 epitope (ELAGIGILTV, SEQ ID NO:12) followed by P2A-HLA-A2/B2M was cloned into a lentiviral vector, which was used to generate MART-1V cancer cells by transduction.

Cas9 and sgRNA targeting P2RY2 (5′-CTGGTCTATTACTACGCCCG-3′ (SEQ ID NO:6) and 5′-TGGGCTGTGTCTGAACGCCG-3′ (SEQ ID NO:7)), CD276 (5′-CACAGGGCAACGCATCCCTG-3′, SEQ ID NO:10), or both were introduced into the cancer cells by lentiviral transduction as described in Example 2. sgRNA(5′-GTATTACTGATATTGGTGGG-3′, SEQ ID NO:11) that does not recognize any sequence in the human genome served as a control. The open reading frames of transcripts of P2RY2 (ENST00000393597.7) or CD276 (ENST00000318443.10), were ordered as a codon optimized gBlock Gene Fragment (Integrated DNA Technologies or Twist Bioscience) and cloned into a lentiviral vector containing a blasticidin selection cassette. Constructs were verified by Sanger sequencing.

MART1-specific CD8+ T cells and MART1 epitope-expressing cancer cells were mixed at a ratio of 1:16 to 1:1. CD33 CAR-T cells and CD33 expressing THP-1 cells were mixed at a ratio of 1:16 to 1:4. CEA CAR-T cells and CEA expressing SW480 cells were mixed at a ratio of 1:1. Next, cells were centrifugated at 1,600 g for 3 min and incubated at 37° C. and 5% CO₂ for 12-72 hours. T cell activation was assessed by surface staining with anti-CD137-BV421 (4-1BB, Biolegend), anti-CD137-PE/Cy7 (4-1BB, Biolegend), anti-CD69-APC/Fire™ 750 (FN50, Biolegend), anti-CD25-AF700 (BC96, Biolegend), anti-CD25-APC (BC96, Biolegend) antibodies and intracellular staining with anti-IL-2-PE/Cy7 (MQ1-17H12, Biolegend), anti-IFN-7-BV421 (4S.B3, Biolegend), anti-TNF-α-BV785 (MAb11, Biolegend), anti-TNF-α-APC (MAb11, Biolegend) antibodies, followed by flow cytometry analysis. For intracellular staining, brefeldin A (Biolegend) was added to the cell culture medium 4 h before cells were harvested. The viability of cancer cells that grow in suspension including THP-1 cells was assessed by flow cytometry-based absolute cell counting using AccuCount beads (Spherotech). For adherent cancer cells including BxPC3, MIA PaCa-2, MDA-MB-231 and MCF7, the cell viability was quantified using CellTiter-Blue Cell Viability Assay (Promega).

### Result

To determine the effects of P2RY2 on T cells and cancer cells in in vitro models of T cell-based immunotherapies, we engineered human T cells that are derived from peripheral blood with a CD33-specific CAR, a CEA-specific CAR, or a MART-1 specific TCR, and assessed T cell activation in the presence of antigen-expressing cancer cells that carry Cas9/sgRNA targeting P2RY, B7-H3, or both, or antigen-expressing cancer cells that overexpress P2RY2 or B7-H3.

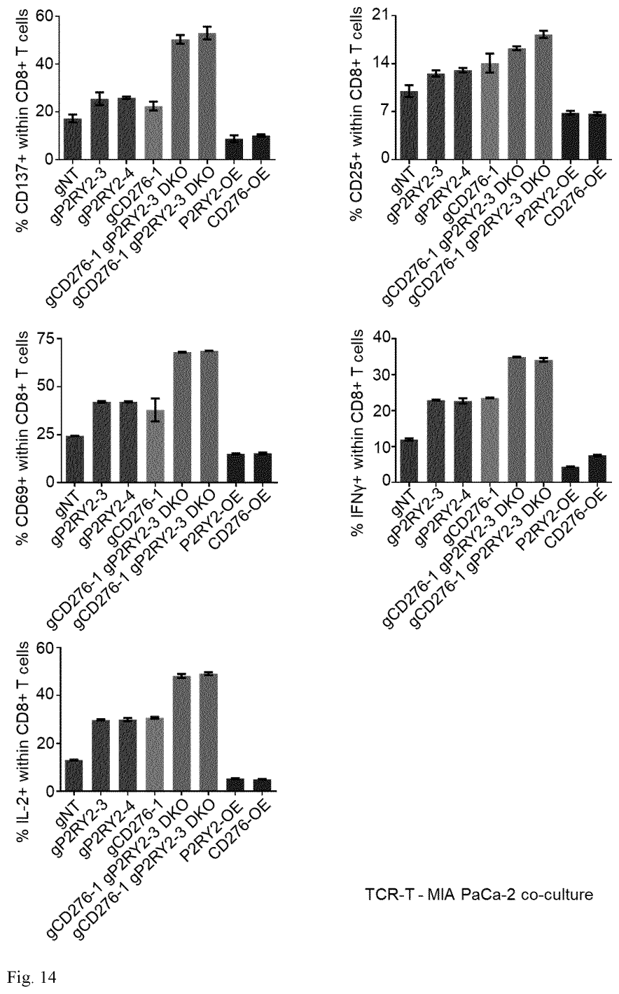

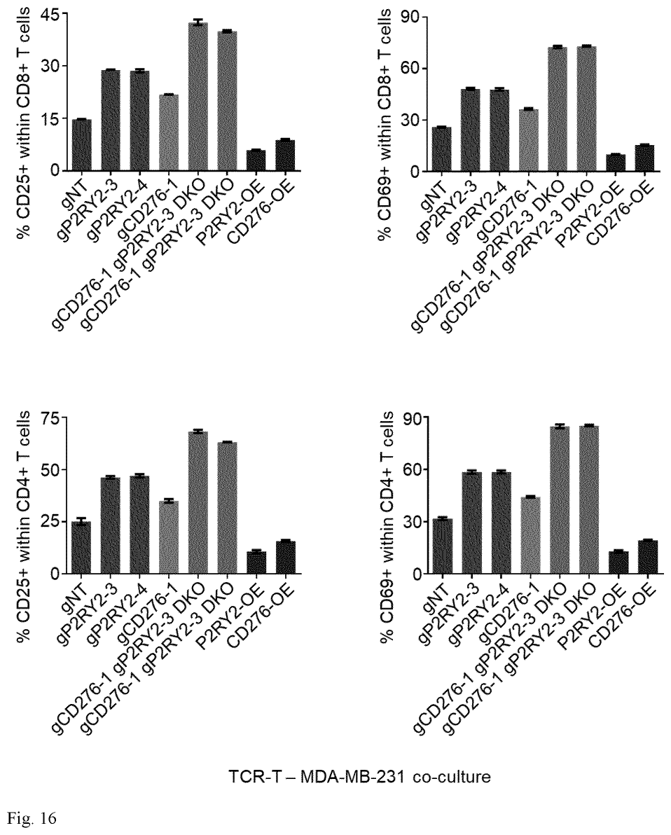

Notably, simultaneous deletion of P2RY2 and B7-H3 in THP-1 increased the expression of these T cell activation markers (CD25, IFNγ, IL-2 and TNFα) to higher levels than individual deletion of P2RY2 or B7-H3 in both CD3+CD8+ and CD3+CD8− T cells (FIG. 4, 5, cf. Example 3). Similar effects were observed in the co-culture of CEA-specific CAR transduced T cells with CEA+SW480 cells (FIG. 12, 13), as well as in the coculture of MART-1-specific TCR transduced T cells with MART-1 epitope transduced BxPC3 cells (FIG. 6, cf. Example 3), MIA PaCa-2 (FIG. 14, 15), or MDA-MB-231 cells (FIG. 16).

In line with these data, P2RY2 or B7-H3 disruption in cancer cells resulted in increased tumor cell killing by tumor-reactive CAR-T or TCR-T cells in myeloid leukemia cell (THP-1), colorectal cancer cell (SW480), pancreatic cancer cell (BxPC3 and MIA PaCa-2), and breast cancer cell (MCF7 and MDA-MB-231) models (FIG. 7, 17, 18). Moreover, combinatorial deletion of P2RY2 and B7-H3 significantly improved the tumoricidal effect of T cells. In contrast, overexpression of P2RY2 or B7-H3 in cancer cells conferred resistance to the tumor-reactive T cells (FIG. 7, 17, 18). These results suggest that P2RY2 suppresses T cell activation through both B7-H3-dependent and B7-H3-independent mechanisms and combinatorial deletion of P2RY2 and B7-H3 further improves T cell-mediated anticancer effects over deletion of either molecule alone.

Consistent with the results shown in FIGS. 8 and 9 above, the P2RY2 inhibitor AR-C 118925XX increased the expression of T cell activation markers CD25, CD69, and IFN-7, whereas the activation of P2RY2 by ATP, non-hydrolysable ATP (Adenosine-5′-(γ-thio)-triphosphate), Diquafosol, or Denufosol led to repressed T cell activation in the coculture of SW480 cells and CEA-specific CAR transduced T cells (FIG. 19). Additionally, when P2RY2 was deleted in SW480 cells, the P2RY2 targeting chemicals had little effect on T cell activation in the same SW480-CEA-specific CAR-T coculture assay. Furthermore, reduced tumor cell viability was observed upon AR-C 118925XX treatment in this assay, while the P2RY2 agonists increased tumor cell survival. Again, this effect is through P2RY2, as evidenced by the fact that all chemicals targeting P2RY2 lost their effects upon P2RY2 depletion (FIG. 20).

Together, these data demonstrate that P2RY2 expression on cancer cells inhibits both T cell activation and T cell-mediated cytotoxicity, and that the modulation of P2RY2 activity can be exploited to modulate T cell activity in immunotherapies.

### REFERENCES

Burnstock et al., Purigergic Signal 9, 491-540 (2013);
Cai et al., Cell Mol Immuno 17(3), 227-236 (2020);
Chen et al., J Immunol 189, 347-355 (2012);
Di Virgilio et al., Oncogene 36, 293-303 (2017);
Doench et al., Nat Biotechnol 34, 184-191 (2016);
Hu et al., Clin Cancer Res 25 (4): 1318-1330 (2019);
Jorritsma et al., Blood 110 (10): 3564-3572. (2007);
Kepp et al., Immunol Rev 280(1): 83-92 (2017);
Lee et al., Cell Res 27, 1034-1045 (2017);
Mezzadra et al., Nature 549, 106-110 (2017);
Pellegatti et al., PLOS one 3(7): e2599 (2008);
Prasad et al., J Immunol 73(4) 2500-2506 (2004);
Suh et al., Nat Immunol 4, 899-906 (2003);
Soltoff et al., J Biol Chem 273 (5) 2653-2660 (1998);
Ueno et al., Eur J Immunol 42, 2343-2353 (2012);
US 2005/0147614A1;
Veenstra et al., Blood 125, 3335-3346 (2015);
WO 99/02501;
WO 2019/178382;
Yim et al., Eur J Cancer 133, 74-85 (2020).

1-15. (canceled)

16. A method for treating a subject suffering from an immune-aggravated disease, the method comprising:
(a) contacting the subject with a P2Y purinoceptor 2 (P2RY2) activity modulator, and
(b) thereby treating the immune-aggravated disease.

17. The method of claim 16, wherein the immune-aggravated disease is cancer or autoimmune disease.

18. The method of claim 16, wherein the treating is T cell immunotherapy.

19. The method of claim 18, wherein the T cell immunotherapy is an adoptive T cell immunotherapy, tumor-infiltrating lymphocyte (TIL) therapy, engineered T cell immunotherapy, chimeric antigen receptor (CAR), and/or recombinant T cell receptor immunotherapy.

20. The method of claim 16, wherein the P2RY2 modulator is a P2RY2 activity decreasing compound, and optionally wherein the P2RY2 activity decreasing compound increases T cell anti-cancer activity in vitro and/or in vivo.

21. The method of claim 18, wherein the T cell immunotherapy is cancer T cell immunotherapy, and optionally further comprising at least one of administration of an immune checkpoint inhibitor, administration of a cytokine, and/or administration of a T cell engager, and identification of the subject as amenable to T cell immunotherapy.

22. The method of claim 20, wherein the P2RY2 activity decreasing compound is a direct P2RY2 activity decreasing compound specifically binding to and inhibiting P2RY2, wherein the direct P2RY2 activity decreasing compound is a small molecule inhibitor, an inhibitor polypeptide, an inhibitor polynucleotide, or a non-polypeptide non-polynucleotide inhibitor macromolecule.

23. The method of claim 22, wherein the small molecule inhibitor is AR-C 118925XX (CAS No: 216657-60-2), and wherein the inhibitor polypeptide is selected from the group consisting of an antibody, an aptamer, an anticalin, and a Designed Ankyrin Repeat Protein (DARPin), and/or wherein the inhibitor polynucleotide is a polynucleotide aptamer.

24. The method of claim 20, wherein the P2RY2 activity decreasing compound is an indirect P2RY2 activity decreasing compound decreasing the amount of P2RY2 in a target cell, wherein the indirect P2RY2 activity decreasing compound is selected from the group consisting of an shRNA, a siRNA, a miRNA agent, an antisense oligonucleotide, a ribozyme, and a CRISPR/Cas oligonucleotide, and optionally a pair of CRISPR/Cas oligonucleotides.

25. The method of claim 16, wherein the P2RY2 activity modulator is administered topically, and optionally intra-tumorally.

26. The method of claim 16, wherein the P2RY2 modulator is a P2RY2 activity increasing compound and wherein the T cell immunotherapy is T cell immunotherapy of autoimmune disease.

27. The method of claim 26, wherein the P2RY2 activity increasing compound is a direct P2RY2 activity increasing compound binding to and activating activity of P2RY2, and
wherein the P2RY2 activity increasing compound is a small molecule activator, an activator polypeptide, an activator polynucleotide, or a non-polypeptide non-polynucleotide activator macromolecule;
preferably wherein the small molecule activator is a nucleotide or a derivative thereof, preferably is ATP or UTP or a derivative thereof, more preferably is MRS 2768 (Uridine-5′-tetraphosphate δ-phenyl ester, CAS No: 1047980-83-5), Uridine-5′-(γ-thio)-triphosphate (CAS No: 1266569-94-1), 4-Thiouridine-5′-O-(β,γ-difluoromethylene)triphosphate (CAS No: 1657025-60-9), Denufosol (CAS No: 211448-85-0) or Diquafosol (CAS No: 59985-21-6).

28. The method of claim 26, wherein the P2RY2 activator is an indirect P2RY2 activator increasing the amount of P2RY2 in a target cell, and wherein the indirect P2RY2 activator is:
(i) a polypeptide comprising a P2RY2 polypeptide;
(ii) a polynucleotide encoding a polypeptide comprising a P2RY2 polypeptide;
(iii) a vector comprising the polynucleotide of (ii);
(iv) a host cell comprising the polynucleotide of (ii) and/or the vector of (iii); or
(v) any combination of (i) to (iv).

29. A method of identifying a subject amenable to T cell immunotherapy comprising:
(A) determining in a sample of the subject the activity (amount) of P2RY2;
(B) comparing the amount determined in step (B) to a reference; and
(C) identifying a subject amenable to T cell immunotherapy based on the comparison of step (C);
preferably, wherein the reference is derived from a (i) subject or group of subjects known to be amenable to immunotherapy or (ii) a subject or group of subjects known not to be amenable to immunotherapy.

30. The method of claim 28, wherein determining the activity comprises determining the amount of P2RY2 and/or of at least one of its downstream signaling molecules, preferably intracellular calcium concentration.

31. A method for identifying a P2RY2 activity modulator, the method comprising:
(I) contacting a host cell with a candidate compound suspected to be a P2RY2 activity modulator;
(II) determining B7-H3 activity in the host cell;
(II) comparing the B7-H3 activity determined in step (II) to a control; and
(IV) identifying a P2RY2 activity modulator based on the comparison in step (II).
